(19) United States
(12) Patent Application Publication
(10) Pub. No.: US 2025/0101428 A1
(43) Pub. Date: Mar. 27, 2025

(54) MODIFICATION PATTERNS FOR SMALL INTERFERING RNA MOLECULES WITH HIGH STABILITY AND GENE SILENCING ACTIVITIES

(71) Applicant: MICROBIO (SHANGHAI) CO. LTD., Shanghai (CN)

(72) Inventors: Yi-Chung CHANG, Shanghai (CN); Chi-Fan YANG, Shanghai (CN); Hui-Yu CHEN, Shanghai (CN); Yi-Fen CHEN, Shanghai (CN)

(21) Appl. No.: 18/715,175

(22) PCT Filed: Dec. 2, 2022
(86) PCT No.: PCT/CN2022/136350

§ 371 Date: May 31, 2024

(30) Foreign Application Priority Data
Dec. 3, 2021 (WO) PCT/CN2021/135465

(51) Int. Cl. C12N 15/113

(52) U.S. Cl. CPC C12N 15/1131; C12N 2310/14; C12N 2310/315; C12N 2310/321

(57) ABSTRACT

Small interfering RNAs (siRNA) having specific modification patterns to improve interference efficiencies thereof. The modification patterns each comprises a combination of 2′-O methyl modification, 2′-O-fluoro-modification, and phosphorothioate (PS) bonds at defined positions in the sense strand and anti-sense strand of each siRNA, and optionally 5′ phosphate modifications.

### CROSS REFERENCE TO RELATED APPLICATIONS

This application claims the benefit of the filing date of International Patent Application No. PCT/CN2021/135465, filed Dec. 3, 2021, the entire contents of which are incorporated by reference herein.

### BACKGROUND OF THE INVENTION

RNA interference (RNAi) is a process of sequence-specific post-transcriptional gene silencing that is mediated by small interfering RNAs (siRNAs). Considerable attention is given to the ability to influence the RNAi to specifically silence the expression of the target genes in order to achieve desired therapeutic effects.

The challenges facing siRNA therapeutics are significant. This is because the inherent properties of siRNAs, such as being polyanionic, vulnerability to nuclease cleavage make clinical application difficult due to poor cellular uptake and rapid clearance. Modifications of nucleotides in siRNA molecules have been employed to enhance stability and resistance to nuclease cleavage. However, many modifications to siRNAs were found to negatively affect their gene silencing activity.

Accordingly, there is a growing need to develop potent siRNAs with desirable stability and gene silencing activity.

### SUMMARY OF THE INVENTION

The present disclosure is based, at least in part, on the development of specific modification patterns for small interfering RNAs to enhance stability and resistance to nuclease digestion without compromising gene silencing activity and to reduce off-target effects.

Accordingly, one aspect of the present disclosure features a modified small interfering RNA (siRNA), comprising a sense strand having 19 nucleotides and an antisense strand having 21 nucleotides. The sense strand, the antisense strand, or both may contain one or more phosphothioate (PS) bonds. In some instances, the sense strand and the antisense stand form a double-stranded siRNA, optionally with a two-nucleotide overhang at the 3′ end of the antisense strand.

In some embodiments, the antisense strand contains (i) 7 to 9 2′-fluoro-modified nucleotides, and (ii) 12 to 14 2′-O-methyl-modified nucleotides. For example, the antisense strand may contain the 2′-fluoro-modified nucleotides at 7 or more of positions 2-12, 14-16, and 18. Alternatively or in addition, the antisense strand may comprise 2′-fluoro-modified nucleotides at position 2 or 3, and position 14, and optionally position 18. In another example, the antisense strand may contain the 2′-O-methyl-modified nucleotides at 12 or more of positions 1, 2-13, and 15-21. Alternatively or in addition, the antisense strand may comprise 2′-O-methyl-modified nucleotides at positions 1, 13, 17 and 19-21. In some instances, the antisense strand may contain PS bonds between positions 1 and 2, positions 2 and 3, positions 19 and 20, and/or positions 20 and 21.

In some instances, the chemically modified siRNAs disclosed herein (e.g., the antisense strand, the sense strand, or both) may further comprise a modified 5′-phosphate. In some examples, the antisense strand comprises the modified 5′ phosphate. In some examples, the modified 5′-phosphate is a E-vinyl phosphonate or a phosphorothiorate.

In any of the modified siRNAs disclosed herein, the sense strand contains (i) 2 to 5 2′-fluoro-modified nucleotides, and (ii) 11 to 16 2-O-methyl-modified nucleotides. For example, the sense strand may contain the 2′-fluoro-modified nucleotides at two or more of positions 5 and 7-11. Alternatively or in addition, the sense strand comprises 2′-fluoro-modified nucleotides at positions 5 and 7-10 or at positions 5, 7, 9, and 11. In another example, the sense strand may contain the 2′-O-methyl-modified nucleotides at 11 or more of positions of 1-6, 8, and 10-19. Alternatively or in addition, the sense strand comprises 2′-O-methyl-modified nucleotides at positions 4, 6, and 11-19. In instances, the sense strand may further comprise 2′-O-methyl-modified nucleotides at positions 8 and 10. In yet other examples, one or more positions 1-3 in the sense strand may contain 2′-O-methyl-modified nucleotides. Alternatively, one or more of positions 1-3 in the sense strand are not modified. Alternatively or in addition, the sense strand may contain a 2′-deoxynucleotide, which optionally is located as position 9. In some instances, the sense strand may contain PS bonds between positions 1 and 2, positions 2 and 3, positions 4 and 5, positions 5 and 6, positions 17 and 18, and/or positions 18 and 19.

In some embodiments, the antisense strand (top) and the sense strand (bottom) of a modified siRNA disclosed herein may have one of the following formulas:

(i)
5′-mffmmffmmffmmffmmfmmm-3′
3′-mmmmmmmmmmdffmmmmmm-5′;
(ii)
5′-mmfmfmfmfmmmmfmfmfmmm-3′
3′-mmmmmmmmmffffmfmmmm-5′;
(iii)
5′-mmfmfmfmfmmmmfmfmfmmm-3′
3′-mmmmmmmmmffffmfmrrr-5′;
(iv)
5′-mfmfmfmffmmmmfmfmfmmm-3′
3′-mmmmmmmmmffffmfmrrr-5′
(v)
5′-mfmffmmffmmmmfmfmfmmm-3′
3′-mmmmmmmmmffffmfmmmm-5′;
(vi)
5′-mfmfmfmfmfmfmfmfmfmmm-3′
3′-mmmmmmmmfmfmfmfmmmm-5′;
(vii)
5′-mfmfmfmfmfmfmfmfmfmmm-3′
3′-mmmmmmmmfmfmfmfmrrr-5″ or
(viii)
5′-mfmfmfmfmfmfmfmfmmmmm-3′
3′-mmmmmmmmfmfmfmfmrrr-3′; and

wherein m represents 2′-O-methyl nucleotide, f represents 2′-fluoro nucleotide, d represents DNA residue, and r represents unmodified nucleotide.

In some examples, the antisense strand (top) and the sense strand (bottom) of a modified siRNA disclosed herein may have the following formulas:

(i)
5′-m*f*fmmffmmffmmffmmfm*m*m-3′
3′-m*m*mmmmmmmmdffmmmm*m*m-5′;
(ii)
5′-m*m*fmfmfmfmmmmfmfmfm*m*m-3′
3′-m*m*mmmmmmmffffmfmm*m*m-5′;
(iii)
5′-m*m*fmfmfmfmmmmfmfmfm*m*m-3′
3′-m*m*mmmmmmmffffm*f*mrrr-5′;
(iv)
5′-m*f*mfmfmffmmmmfmfmfm*m*m-3′
3′-m*m*mmmmmmmffffm*f*mrrr-5′
(v)
5′-m*f*mffmmffmmmmfmfmfm*m*m-3′
3′-m*m*mmmmmmmffffmfmm*m*m-5′;
(vi)
5′-m*f*mfmfmfmfmfmfmfmfm*m*m-3′
3′-m*m*mmmmmmfmfmfmfmm*m*m-5′;
(vii)
5′-m*f*mfmfmfmfmfmfmfmfm*m*m-3′
3′-m*m*mmmmmmfmfmfm*f*mrrr-5″ or
(viii)
5′-m*f*mfmfmfmfmfmfmfmmm*m*m-3′
3′-m*m*mmmmmmfmfmfm*f*mrrr-3′; and

wherein m represents 2′-O-methyl nucleotides, f represents 2′-fluoro nucleotides, d represents DNA residue, r represents unmodified nucleotides, and * represents PS bonds.

In some embodiments, the sense strand of any of the modified siRNAs disclosed herein may be conjugated to a targeting moiety, for example, conjugated to N-acetylgalactosamine (GalNAc). Also provided herein are uses of any of the modified siRNAs or the pharmaceutical composition comprising such for manufacturing a medicament for use in silencing expression of the target gene and treating diseases associated with the target gene.

Further, the instant disclosure also provides a pharmaceutical composition comprising any of the modified siRNAs as disclosed herein and a pharmaceutically acceptable carrier. Such a pharmaceutical composition can be for use in silencing a gene targeted by the siRNA.

In other aspects, provided herein is a method for modulating gene expression, comprising contacting cells with any of the modified siRNAs disclosed herein or the pharmaceutical composition comprising such. In some embodiments, the contacting step can be performed by administering the modified siRNA or the pharmaceutical composition to a subject having a disease associated with the gene of interest.

The details of one or more embodiments of the disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram illustrating exemplary modification patterns for small interfering RNAs (siRNAs). m: 2′-O-methyl. f: 2′-O fucosyl. Bars between nucleotides represent PS internucleotide linkages. The PS bonds can present at either or both ends of the siRNA molecules.

FIG. 2A-2B include diagrams showing 5′-phosphate modifications and the associated interference efficacy. FIG. 2A: chemical structure of exemplary 5′-phosphate modifications 5′-E-vinylphosphonate and 5′-phosphorothioate. FIG. 2B: a bar graph showing improved gene silencing activities of anti-HBx siRNAs with 5′-E-vinylphosphonate modification in the antisense strand (X32-G25S-PS6-VP), or 5′-phosphorothioate modification in the antisense strand (X32-G25S-PS6-5S), as compared with the siRNA counterpart without the 5′-phosphate modification (X32-G25S-PS6).

### DETAILED DESCRIPTION OF THE INVENTION

RNA interference or “RNAi” is a process in which double-stranded RNAs (dsRNA) block gene expression when it is introduced into host cells. (Fire et al. (1998) Nature 391, 806-811). Short interfering RNA molecules (siRNA) are commonly used in RNAi to inhibit expression of a target gene, thereby achieving desired therapeutic effects.

siRNAs are double-stranded RNAs, an anti-sense strand and a sense strand, which contain complementary sequences and form the double-stranded structure. At least part of the anti-sense strand is complementary to a region within a target mRNA for blocking expression of the mRNA via RNAi. Each strand of a siRNA molecule may have 19-23 nucleotides. In some instances, each strand may have phosphorylated 5′ ends and hydroxylated 3′ ends. In some instances, the anti-sense strand may have a couple overhanging nucleotides (e.g., 1 or 2). When the siRNA is transfected into a cell, it is incorporated into the RNA-induced silencing complex (RISC), which includes the core protein Argonaute (AGO). Subsequently, the siRNA is unwound into single-stranded RNAs and the antisense strand remains associated with AGO to form an active RISC, whereas the sense strand is degraded. The antisense strand forms base-pairings with a target transcript (mRNA), and AGO cleaves the target to silence its function (gene expression).

While siRNAs are promising therapeutic agents for regulating expression of target genes to achieve desired therapeutic effects, RNA molecules are generally vulnerable to nuclease digestion in vivo, which would impair their clinical applications. Introducing modifications to RNA molecules is a common approach to enhance stability of RNA therapeutics. However, positions of modifications and/or levels of modifications often impact gene silencing activity of a resultant siRNA. It is therefore of great interest to design proper modification patterns for siRNAs to enhance stability and/or resistance to nuclease digestion, while maintain desirable gene silencing activity. Such modifications can also reduce off-target effects when compared to an unmodified siRNA counterpart.

Accordingly, provided herein, in some aspects, are specific modification patterns (e.g., a combination of specific nucleotide modifications and phosphothioate (PS) bonds at defined positions in both the sense strand and the anti-sense stand of an siRNA) for siRNAs, pharmaceutical compositions comprising such modified siRNAs, and uses thereof for silencing expressing of a target gene.

Unless otherwise explicitly stated, the position of a nucleotide in a nucleic acid chain (e.g., sense strand or antisense strand) as disclosed herein refers to the position from the 5′ end of that nucleic acid chain, i.e., with the 5′ end nucleotide as Position 1.

I. Modification Patterns for siRNAs

In some aspects, this present disclosure relates to specific modification patterns of small interfering nucleic acid molecules (siRNA). A modification pattern refers to the modification profile, including specific nucleotide modifications (e.g., 2′-O-methyl-modified nucleotides, 2′-fluoro-modified nucleotides, or a combination thereof) at defined positions in the sense stand and antisense strand, and/or PS bonds at defined positions in the sense and antisense strand. In some instances, the antisense strand and/or the sense strand of any of the modified siRNAs disclosed herein may further comprise a 5′-phosphate modification, e.g., those disclosed herein. siRNAs having such modification patterns show similar or better gene silencing activity as compared with their unmodified counterparts. Such modified siRNAs are also expected to be more stable and/or resistance to nuclease digestion relative to the unmodified counterparts.

(A) siRNA Molecules:

The disclosure relates to modified siRNA molecules, which are double-stranded RNAs capable of inducing gene silencing via the RNAi pathway against the target gene transcript at a high level and also have high stability and/or resistance to nuclease digestion. Such modified siRNAs are expected to be more therapeutically effective as compared with the unmodified counterparts.

The modified siRNA molecule comprises a sense strand and an antisense strand, which of which has a specific modification pattern. In any of the modified siRNA molecules disclosed herein, the antisense strand may contain 15-30 nucleotides in length, e.g., 18-25 or 19-23 nts in length. In one example, the antisense strand includes 21 nts in length. In another example, the antisense strand includes 23 nts in length. The sense strand or a portion thereof is complementary (completely or partially) to the antisense strand or a portion thereof. In some instances, the sense strand has the same length as the antisense strand. In other instances, the sense strand is shorter than the antisense strand (e.g., by 1-5 nt such as by 1 nt, 2 nt, 3 nt, 4 nt, or 5 nt). In that case, the antisense strand may have overhang (e.g., 1-5 nts) at the 5′ end and/or at the 3′ end.

In some embodiments, the sense strand contains 19-nucleotides and the antisense strand contains 21 nucleotides. The sense strand and the antisense strand form a double stranded siRNA molecule, which, in some instances, has a two-nucleotide overhand at the 3′ end of the antisense strand.

In some embodiments, the antisense strand in a modified siRNA disclosed herein may comprise 7-9 (e.g., 7, 8, or 9) 2′-fluoro-modified nucleotides and 12 to 14 (e.g., 12, 13, and 14) 2′-O-methyl-modified nucleotides. Alternatively or in addition, the sense strand in the modified siRNA may contain 2-5 (e.g., 2, 3, 4, or 5) 2′-fluoro-modified nucleotides and 11-16 (e.g., 11, 12, 13, 14, 15, or 16) 2′-O-methyl-modified nucleotides. Such a modified siRNA may further comprise one or more PS bonds in the sense strand and/or in the antisense strand.

In some examples, the antisense strand includes 21 nucleotides. Such an antisense strand may have 2′-fluoro-modified nucleotides at 7 or more of positions 2-12, 14-16, and 18. For example, the antisense strand may have 2′-fluoro-modified nucleotides at positions 2 or 3 and position 14. In some instances, the antisense strand have 2′-fluoro-modified nucleotides at positions 2, 14 and 18. Alternatively, the antisense strand may have 2′-fluoro-modified nucleotides at positions 2, 14 and 18. In specific examples, the antisense strand may have 2′-fluoro-modified nucleotides at: (i) positions 2, 3, 6, 7, 10, 11, 14, 15, and 18; (ii) positions 3, 5, 7, 9, 14, 16, and 18; (iii) positions 3, 5, 7, 9, 14, 16, and 18; (iv) positions 2, 4, 6, 8, 9, 14, 16, and 18; (v) 2, 4, 5, 8, 9, 14, 16, and 18; (vi) positions 2, 4, 6, 8, 10, 12, 14, 16, and 18; or (vii) positions 2, 4, 6, 8, 10, 12, 14, or 16.

Further, the antisense strand may have 2′-O-methyl-modified nucleotides at 12 or more of positions 1, 2-13, and 15-21. For example, the antisense strand may comprise 2′-O-methyl-modified nucleotides at least at positions 1, 13, 17, and 19-21. In specific examples, the antisense strand may have 2′-O-methyl-modified nucleotides at: (i) positions 1, 4, 5, 8, 9, 12, 13, 16, 17, and 19-21; (ii) positions 1, 2, 4, 6, 8, 10-13, 15, 17, and 19-21; (iii) positions 1, 2, 4, 6, 8, 10-13, 15, 17, and 19-21; (iv) 1, 3, 5, 7, 10-13, 15, 17, and 19-21; (v) positions 1, 3, 6, 7, 10-13, 15, 17, and 19-21; (vi) 1, 3, 5, 7, 9, 11, 13, 15, 17, and 19-21; (vii) positions 1, 3, 5, 7, 9, 11, 13, 15, and 17-21.

Alternatively or in addition, the antisense strand may contain PS bonds between positions 1 and 2, positions 2 and 3, positions 19 and 20, positions 20 and 21, or a combination thereof. In specific examples, the antisense strand may have PS bonds at between positions 1 and 2, between positions 2 and 3, between positions 19 and 20, and between positions 20-21.

In some examples, the sense strand of any of the modified siRNAs disclosed herein may include 19 nucleotides. Such a sense strand may have 2′-fluoro-modified nucleotides at two or more of positions 5 and 7-11. For example, the sense strand may comprise 2′-fluoro-modified nucleotides at positions 5 and 7-10. Alternatively, the sense strand may comprise 2′-fluoro-modified nucleotides at positions 5, 7, 9, and 11. In specific examples, the sense strand may have 2′-fluoro-modified nucleotides at: (i) positions 7 and 8; (ii) positions 5 and 7-10; or (iii) positions 5, 7, 9, and 11.

Further the sense strand of any one of the modified siRNAs disclosed herein may have 2′-O-methyl-modified nucleotides at 11 or more of positions of 1-6, 8, and 10-19. For example, the sense strand may comprise 2′-O-methyl-modified nucleotides at positions 4, 6, and 11-19. In some instances, the sense strand may further comprise 2′-O-methyl-modified nucleotides at positions 8 and 10. In other examples, the sense strand may comprise 2′-O-methyl-modified nucleotides at positions 1-3. Alternatively, the sense strand may comprise unmodified nucleotides at one or more of positions 1-3 (e.g., all of positions 1-3). In specific examples, the sense strand may contain 2′-O-methyl-modified nucleotides at: (i) positions 1-6 and 10-19; (ii) positions 1-4, 6, and 11-19; (iii) positions 4, 6, and 11-19; or (iv) positions 4, 6, 8, 10 and 12-19.

Alternatively or in addition, the sense strand may contain PS bonds between positions 1 and 2, positions 2 and 3, positions 4 and 5, positions 5 and 6, positions 17 and 18, positions 18 and 19, or a combination thereof. In some examples, the sense strand may contain PS bonds between positions 1 and 2, between positions 2 and 3, between positions 17 and 18, and between positions 18 and 19. In other examples, the sense strand may contain PS bonds between positions 4 and 5, between positions 5 and 6, between positions 17 and 18, and between positions 18 and 19.

### (B) 5′-Phosphate Modifications

In some embodiments, any of the modified siRNAs disclosed herein may further comprise a 5′-phosphate modification at the sense strand, at the antisense strand, or both. In some examples, the antisense strand comprises a 5′-phosphate modification, while the sense strand is free of such a modification. In some examples, the sense strand comprises a 5′-phosphate modification, while the antisense strand is free of such a modification. In some examples, both the antisense strand and the sense strand comprise a 5′-phosphate modification.

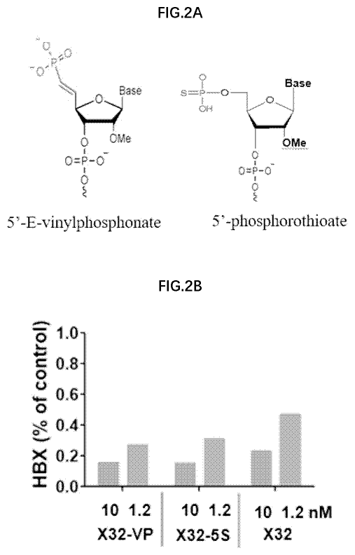

5′-phosphate modifications refer to one or more substitutions of a suitable moiety in the 5′-end phosphate residue. Examples include, but are not limited to, E-vinyl phosphonate (5′-E-VP), Z-vinyl phosphonate (5′-Z-VP), 5′-methyl phosphonate, a 5′-C-methyl analog, and a phosphorothioate. In one example, the 5′-phosphate modification is 5′-E-VP. In another example, the 5′-phosphate modification is phosphorothioate. See FIG. 2B.

### (C) Other Modifications

In some embodiments, the modified siRNA disclosed herein contain only the 2′-fluoro-modified nucleotides, the 2′-O-methyl-modified nucleotides, and PS bonds, optionally the 2′deoxynucleotide in the sense strand, as disclosed herein with no further modifications.

Alternatively, the antisense strand, the sense strand, or both of the modified siRNAs can further comprise other modifications such as sugar modifications, nucleobase modifications, backbone modifications, or a combination thereof. For example, the sense strand may contain a 2′-deoxynucleotide. When the sense strand has 19 nucleotides, the 2′-deoxynucleotide may be located as position 9. Such modifications may confer one or more desirable properties, for example, enhanced cellular uptake, improved affinity to the target nucleic acid, increased in vivo stability, enhance in vivo stability (e.g., resistant to nuclease degradation), and/or reduce immunogenicity.

In one example, the modified siRNAs disclosed herein (e.g., in the sense strand and/or antisense strand) may have a modified backbone at positions different from the PS internucleotide bonds, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In some examples, the modified siRNAs disclosed herein do not include any backbone modifications, except for the PS internucleotide bonds disclosed herein.

In another example, the modified siRNAs disclosed herein (e.g., in the sense strand and/or antisense strand) include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, the modified siRNAs disclosed herein (e.g., in the sense strand and/or antisense strand) include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the interfering RNA molecules to their targeting sites. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Alternatively or in addition, the modified siRNAs disclosed herein (e.g., in the sense strand and/or antisense strand) may comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form duplexes. LNA nucleotides can be used in any of the modified siRNAs disclosed herein. In some examples, up to 50% (e.g., 40%, 30%, 20%, or 10%) of the nucleotides in an interfering RNA are LNAs.

(D) Exemplary Modification Patterns of siRNAs

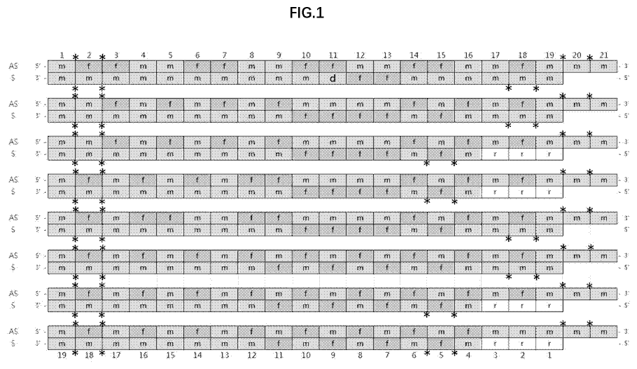

In some examples, the modified siRNAs disclosed herein may have one of the 2′-fluoro and 2′-O-methyl modification patterns in the sense strand illustrated in FIG. 1. In other examples, the modified siRNAs disclosed herein may have one of the 2′-fluoro and 2′-O-methyl modification patterns in the antisense strand illustrated in FIG. 1. In specific examples, the modified siRNAs disclosed herein may have one of the 2′-fluoro and 2′-O-methyl modification patterns in the sense strand and the antisense strand illustrated in FIG. 1. Such a modified siRNA may further comprise the modification pattern of the PS bonds for the sense strand and/or the antisense strand as also illustrated in FIG. 1. See also the formulas of the antisense and sense strands disclosed herein. Formulas with no indication of PS bonds or other modifications of backbones are meant to encompass both strands having no backbone modification and strands with any type of backbone modifications.

Other specific exemplary modified siRNAs are provided in Examples 1 and 2 below, which are also within the scope of the present disclosure.

In some aspects, any of the modified siRNA molecules described herein may be conjugated to a ligand (targeting moiety) or encapsulated into vesicles that can facilitate the delivery of the modified siRNA to desired cells/tissues and/or facilitate cellular uptake. Suitable ligands include, but are not limited to, carbohydrate, peptide, antibody, polymer, small molecule, cholesterol and aptamer. For example, one or more GalNAc moieties (e.g., a tri-GalNAc moiety) may be used as the targeting moiety for delivering the modified siRNAs to liver cells.

### (E) Target Genes

The modified siRNA as disclosed herein is for use to suppress expression of a target gene, the transcript of which (mRNA) contains a region that is complementary to the antisense strand in the modified siRNA. Accordingly, the sequence of the antisense and sense strands can be designed based on the mRNA sequence of the target gene. In some instances, the antisense strand may be completely complementary to a target region within the mRNA of the target gene. In other instances, the antisense strand may be partially complementary to a target region within the mRNA of the target gene (e.g., contain one or more mismatches or gaps) as long as the level of complementarity is sufficient for base-pairing with the target region, which is within the knowledge of a skilled person in the art.

In some embodiments, the target gene of the modified siRNA disclosed herein is a pathogenic gene. For example, the target gene may be a gene of a pathogen, e.g., a virus, a bacterium, or a fungus. In other examples, the target gene is involved in a disease or disorder, for example, cancer, an immune disorder (e.g., an autoimmune disease), metabolic disorders or diseases, cardiovascular disorders or diseases and other inherited disorders or diseases.

In some examples, the modified siRNA silences expression of a target gene involved in cancer. Exemplary cancer-associated target genes include, but are not limited to, HIF1A, HIF2, IGF1R, VEGF, EREG, KRAS, ALK, BRAF, NRAS, STAT3, CDH2, KIFL1, PIK3CA, Src, RAS, RAF, and TP53.

In some examples, the modified siRNA silences expression of a target gene involved in fibrosis. Exemplary fibrosis-associated target genes include, but are not limited to, HIF1A, HIF1B, HIF2, TGF-β1, and CTGF.

In some examples, the modified siRNA silences expression of a target gene involved in a metabolic disease. Exemplary metabolic-associated target genes include, but are not limited to, AGT, ApoC-III, and apoB.

In some examples, the modified siRNA silences expression of a target gene involved in an immune disease (e.g., an autoimmune disease). Exemplary immune disease-associated target genes include, but are not limited to, GATA-3, CCR3, TGF-β1, IL-6, TNF-α, IFN-γ, IL-1β, CCL2, and CCL10.

In some examples, the modified siRNA may target the genome of a virus or the mRNA expressed therefrom, for example, the genomic and/or mRNA of a coronavirus such as SARS-COV2. In some instances, the modified siRNA may silence expression of a viral protein, for example, a hepatitis B viral protein (e.g., HBx).

### III. Pharmaceutical Compositions

Any of the modified siRNA molecule as disclosed herein may be formulated into a suitable pharmaceutical composition. The pharmaceutical compositions as described herein can further comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some examples, the pharmaceutical composition described herein includes excipients that may include, but not limited to, trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane, purified water, ethanol, propylene glycol, glycerin, PEG (e.g., PEG400, PEG 600, PEG 800 and PEG 1000), sorbitan trioleate, soya lecithin, lecithin, oleic acid, Polysorbate 80, magnesium stearate and sodium lauryl sulfate, methylparaben, propylparaben, chlorobutanol, benzalkonium chloride, cetylpyridinium chloride, thymol, ascorbic acid, sodium bisulfite, sodium metabisulfite, EDTA, sodium hydroxide, tromethamine, ammonia, HCl, H₂SO₄, HNO₃, citric acid, CaCl₂), CaCO₃, sodium citrate, sodium chloride, disodium EDTA, saccharin, menthol, ascorbic acid, glycine, lysine, gelatin, povidone K25, silicon dioxide, titanium dioxide, zinc oxide, lactose, lactose monohydrate, lactose anhydrate, mannitol, and dextrose.

In other examples, the pharmaceutical composition described herein can be formulated in sustained-release format. Suitable examples of sustained-release preparations include semipermeable matrices of solid hydrophobic polymers which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle or a sealed container to be manually accessed.

The pharmaceutical compositions described herein can be in unit dosage forms such as solids, solutions or suspensions, or suppositories, for administration by inhalation or insufflation, intrathecal, intrapulmonary or intracerebral routes, oral, parenteral or rectal administration.

For preparing solid compositions, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as powder collections, tablets, pills and capsules. This solid preformulation composition is then subdivided into unit dosage forms of the type described above containing a suitable amount of the active ingredient in the composition.

Suitable surface-active agents include, in particular, non-ionic agents, such as polyoxyethylenesorbitans (e.g., TWEEN® 20, 40, 60, 80 or 85) and other sorbitans (e.g., SPAN 20, 40, 60, 80 or 85). Compositions with a surface-active agent will conveniently comprise between 0.05 and 5% surface-active agent, e.g., between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™, and LIPIPHYSAN™ The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. In some embodiments, the compositions are composed of particle sized between 10 nm to 100 mm.

Compositions in preferably sterile pharmaceutically acceptable solvents may be nebulized by use of gases. Nebulized solutions may be breathed directly from the nebulizing device or the nebulizing device may be attached to a face mask, tent, endotracheal tube and/or intermittent positive pressure breathing machine (ventilator). Solution, suspension or powder compositions may be administered, preferably orally or nasally, from devices which deliver the formulation in an appropriate manner.

In some embodiments, any of the modified siRNA molecule can be encapsulated or attached to a liposome, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, any of the modified siRNA molecule may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

Any of the pharmaceutical compositions comprising the modified siRNA molecule disclosed herein may further comprise a component that enhances transport of the composition from endosomes and/or lysosomes to cytoplasm. Examples include a pH-sensitive agent (e.g., a pH-sensitive peptide).

In some embodiments, any of the pharmaceutical compositions herein may further comprise a second therapeutic agent based on the intended therapeutic uses of the composition.

### IV. Suppressing Target Gene Expression

Any of the modified siRNA molecules disclosed herein may be used to suppress expression of the target gene either in vivo or in vitro.

To practice the method disclosed herein, an effective amount of the pharmaceutical composition described herein that comprise the modified siRNA molecule can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, intratumoral, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution.

As used herein, “an effective amount” refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of the modified siRNAs may be appropriate. Various formulations and devices for achieving sustained release are known in the art.

Generally, for administration of any of the modified siRNA molecules described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the siRNAs, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen can vary over time.

In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of the modified siRNA as described herein will depend on the type and severity of the disease/disorder, whether the modified siRNA is administered for preventive or therapeutic purposes, previous therapy, the patient's clinical history and response to the antagonist, and the discretion of the attending physician. A clinician may administer the modified siRNA molecule until a dosage is reached that achieves the desired result. In some embodiments, the desired result is a decrease in tumor burden, a decrease in cancer cells, or increased immune activity.

Methods of determining whether a dosage resulted in the desired result would be evident to one of skill in the art. Administration of one or more modified siRNA molecule can be continuous or intermittent, depending, for example, upon the recipient's physiological condition, whether the purpose of the administration is therapeutic or prophylactic, and other factors known to skilled practitioners. The administration of the modified siRNA molecule may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intratumoral, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some embodiments, the composition can be administered via a nasal route, for example, intranasal spray, nasal spray, or nasal drops.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, the modified siRNAs can be administered by the drip method, whereby a pharmaceutical formulation containing the interfering RNA and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the modified siRNAs disclosed herein, can be dissolved and administered in a pharmaceutical excipient such as Water-for-Injection, 0.9% saline, or 5% glucose solution.

In one embodiment, the modified siRNA molecule is administered via site-specific or targeted local delivery techniques. Examples of site-specific or targeted local delivery techniques include various implantable depot sources of the therapeutic RNA molecule or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No.

Targeted delivery of therapeutic compositions containing a polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

In some embodiments, any of the modified siRNA molecule, or a pharmaceutical composition comprising such can be administered by pulmonary delivery system, that is, the active pharmaceutical ingredient is administered into lung. The pulmonary delivery system can be an inhaler system. In some embodiment, the inhaler system is a pressurized metered dose inhaler, a dry powder inhaler, or a nebulizer. In some embodiment, the inhaler system is with a spacer.

In some embodiment, the pressurized metered dose inhaler includes a propellent, a co-solvent, and/or a surfactant. In some embodiment, the propellent is selected from the group comprising of fluorinated hydrocarbons such as trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane. In some embodiment, the co-solvent is selected from the group comprising of purified water, ethanol, propylene glycol, glycerin, PEG400, PEG 600, PEG 800 and PEG 1000. In some embodiment, the surfactant or lubricants is selected from the group comprising of sorbitan trioleate, soya lecithin, lecithin, oleic acid, Polysorbate 80, magnesium stearate and sodium lauryl sulfate. In some embodiment, the preservatives or antioxidants is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some embodiment, the pH adjustments or tonicity adjustments is selected from the group comprising of sodium oxide, tromethamine, ammonia, HCl, H₂SO₄, HNO₃, citric acid, CaCl₂), CaCO₃.

In some embodiment, the dry powder inhaler includes a disperse agent. In some embodiment, the disperse agent or carrier particle is selected from the group comprising of lactose, lactose monohydrate, lactose anhydrate, mannitol, dextrose which their particle size is about 1-100 μm.

In some embodiment, the nebulizer may include a co-solvent, a surfactant, lubricant, preservative and/or antioxidant. In some embodiment, the co-solvent is selected from the group comprising of purified water, ethanol, propylene glycol, glycerin, PEG (e.g., PEG400, PEG600, PEG800 and/or PEG 1000). In some examples, the surfactant or lubricant is selected from the group comprising of sorbitan trioleate, soya lecithin, lecithin, oleic acid, magnesium stearate and sodium lauryl sulfate. In some examples, the preservative or antioxidant is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some examples, the nebulizer further includes a pH adjustment or a tonicity adjustment, which is selected from the group comprising of sodium oxide, tromethamine, ammonia, HCl, H₂SO₄, HNO₃, citric acid, CaCl₂), CaCO₃.

In some embodiments, a DNA molecule capable of producing an anti-HIF1a interfering RNA or a pharmaceutical composition comprising such may be used for silencing HIF1a expression. A pharmaceutical composition comprising such a DNA molecule (e.g., a vector) may be administered to a subject in need of the treatment in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

In some embodiments, a DNA molecule capable of producing an anti-SARS-COV interfering RNA or a pharmaceutical composition comprising such may be used for silencing a SARS-COV2 gene. A pharmaceutical composition comprising such a DNA molecule (e.g., a vector) may be administered to a subject in need of the treatment in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The term “about” or “approximately” used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

A subject to be treated by any of the modified siRNA molecules may have or suspected of having a disease associated with the target gene, suppressing of which can be achieved by the modified siRNA molecules (see Target Genes disclosed above). The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, monkey etc. A human subject who needs the treatment may be a human patient having, at risk for, or suspected of having a target disease/disorder, such as tumor.

Any of the modified siRNA molecules disclosed herein may be used for treating a disease or disorder associated with the target gene. Exemplary diseases include, but are not limited to, cancer, fibrosis, a metabolic disease, a cardiovascular disease, an immune disease, or an inheritance disorder.

Any of the modified interfering RNAs as disclosed herein may be used for treating a disease or disorder associated with gene to which the interfering RNA targets. Examples include, but are not limited to, solid tumors, cancers, ischemic heart disease, congestive heart failure, acute lung injury, pulmonary hypertension, pulmonary fibrosis, chronic obstructive pulmonary disease, acute liver failure, liver fibrosis and cirrhosis, acute kidney injury, chronic kidney disease, obesity and diabetes mellitus.

Any of the modified siRNAs disclosed herein may be used in a combined therapy with one or more additional therapeutic agents for treating the target disease. The term combination therapy, as used herein, embraces administration of these agents (e.g., the modified siRNA molecule and the additional therapeutic agents) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route including, but not limited to, oral routes, intravenous routes, intramuscular, intratumoral, subcutaneous routes, direct absorption through mucous membrane tissues, and pulmonary delivery routes. The agents can be administered by the same route or by different routes. For example, a first agent can be administered by pulmonary delivery routes, and a second agent can be administered intravenously.

As used herein, the term “sequential” means, unless otherwise specified, characterized by a regular sequence or order, e.g., if a dosage regimen includes the administration of a composition and an antiviral agent, a sequential dosage regimen could include administration of the composition before, simultaneously, substantially simultaneously, or after administration of the antiviral agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the agents described herein.

Combination therapy can also embrace the administration of the agents described herein, in further combination with other biologically active ingredients and non-drug therapies. It should be appreciated that any combination of a composition described herein and a second therapeutic agent may be used in any sequence for treating a target disease.

Treatment efficacy for a target disease/disorder can be assessed by methods well-known in the art.

In some embodiments, any of the modified siRNAs may be used to suppress expression of the target gene in vitro. To perform such a method, the modified siRNA (e.g., via an encoding nucleic acid such as a vector) may be in contact with cells cultured in vitro, e.g., for research purposes such as for studying disease mechanisms and/or for drug candidate validation.

### V. Kits

The present disclosure can be used alone or as a component of a kit having at least one of the reagents necessary to carry out the in vitro or in vivo introduction of siRNA to test samples and/or subjects.

For example, preferred components of the kit include the modified siRNA molecule of the disclosure and a vehicle that promotes introduction of the siRNA into cells of interest as described herein (e.g., using lipids and other methods of transfection known in the art, see for example Beigelman et al, U.S. Pat. No. 6,395,713).

The kit can also be used for target validation, such as in determining gene function and/or activity, or in drug optimization, and in drug discovery (see for example Usman et al., U.S. Ser. No. 60/402,996). Such a kit can also include instructions to allow a user of the kit to practice the disclosure. Such kits can optionally include one or more of the second therapeutic agents as also described herein.

In some embodiments, the kit can comprise instructions for use in accordance with any of the methods described herein. The kit may further comprise a description of selecting an individual suitable for treatment based on identifying whether that individual has the disease or is at risk for the disease.

The instructions relating to the use of the modified siRNA molecule to achieve the intended therapeutic effects generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk, or QR code) are also acceptable.

The label or package insert may indicate that the composition is used for the intended therapeutic utilities. Instructions may be provided for practicing any of the methods described herein.

The kits of this disclosure are in suitable packaging. Suitable packaging includes, but is not limited to, chambers, vials, bottles, jars, flexible packaging (e.g., sealed Mylar or plastic bags), and the like. Also contemplated are packages for use in combination with a specific device, such as an inhaler, nebulizer, ventilator, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Chemically Modified siRNAs Targeting HIF1A and Interference Efficiencies Thereof

To identify chemical modification patterns of siRNAs that provide superior interference efficiencies of the target gene (HIF-1A in this example), two anti-HIF-1A siRNAs, AI3 and AT9, were each modified in manners shown in Tables 1 and 2 below.

TABLE 1
Modified HIF-1A AI3 siRNAs and Activities Thereof for inhibiting HIF-1A

| siRNA | | | | Expression |
|---|---|---|---|---|
| no. | Strand | Sequence (5′-3′) | Seq ID no. | level (%) |
| AI3 | S | AGGCCACAUUCACGUAUAA | 1 | 47.52% |
| | AS | UUAUACGUGAAUGUGGCCUGU | 2 | |
| AI3- | S | mA*mG*mGmCfCmAfCmAfUmUfCmAmCmGmUmAmUmAmA | 3 | 67.67% |
| G1S | AS | mU*fU*mAfUmAfCmGfUmGfAmAfUmGfUmGfGmCfCmU*mG* | 4 | |
| | | mU | | |
| AI3- | S | mA*mG*mGmCmCfAmCfAdTfUmCmAmCmGmUmAmUmAmA | 5 | 77.38% |
| G2S | AS | mU*mU*fAmUfAmCfGmUfGmAfAmUfGmUfGmGfCmCfU*mG* | 6 | |
| | | mU | | |
| AI3- | S | mA*mG*mGmCmCfAfCmAdTfUfCmAmCmGmUmAmUmAmA | 7 | 83.51% |
| G3S | AS | mU*mU*fAfUmAmCfGfUmGmAfAfUmGmUfGfGmCmCfU*mG* | 8 | |
| | | mU | | |
| AI3- | S | mA*mG*mGmCmCmAfCfAdTmUmCmAmCmGmUmAmUmAmA | 9 | 38.87% |
| G4S | AS | mU*fU*fAmUmAfCfGmUmGfAfAmUmGfUfGmGmCfCmU*mG* | 10 | |
| | | mU | | |
| AI3- | S | mA*mG*mGmCfCmAmCfAfUmUmCmAmCmGmUmAmUmAmA | 11 | 77.38% |
| G5S | AS | mU*mU*fAmUfAfCmGmUfGfAmAmUfGfUmGmGfCfCmU*mG* | 12 | |
| | | mU | | |
| AI3- | S | mA*mG*mGmCfCfAmCmAfUfUmCmAmCmGmUmAmUmAmA | 13 | 78.28% |
| G6S | AS | mU*fU*mAfUfAmCmGfUfGmAmAfUfGmUmGfGfCmCmU*mG* | 14 | |
| | | mU | | |
| AI3- | S | mA*mG*mGfCmCfAfCfAfUmUmCmAmCmGmUmAmUmAmA | 15 | 75.96% |
| G7S | AS | mU*fU*mAfUmAfCmGfUmGfAmAmUmGmUfGmGfCmCmU*m | 16 | |
| | | G*mU | | |
| AI3- | S | mA*mG*mGmCfCmAfCfAfUfUmCmAmCmGmUmAmUmAmA | 17 | 49.94% |
| G8S | AS | mU*mU*fAmUfAmCfGmUfGmAmAmUmGfUmGfGmCfCmU*m | 18 | |
| | | G*mU | | |
| AI3- | S | mA*mG*mGmCmCfAfCmAfUfUmCmAmCmGmUmAmUmAmA | 19 | 86.85% |
| G9S | AS | mU*fU*mAfUfAmCfGmUfGmAmAfUmGmUfGmGfCmCmU*mG | 20 | |
| | | *mU | | |
| AI3- | S | mA*mG*mGmCfCfAfCfAfUmUmCmAmCmGmUmAmUmAmA | 21 | 75.44% |
| G10S | AS | mU*fU*mAfUmAfCmGfUmGfAmAmUmGmUmGfGmCfCmU*m | 22 | |
| | | G*mU | | |
| AI3- | S | rArGrGmC*fC*mAfCmAfUmUfCmAmCmGmUmAmUmAmA | 23 | 49.77% |
| GIA | AS | mU*fU*mAfUmAfCmGfUmGfAmAfUmGfUmGfGmCfCmU*mG* | 24 | |
| | | mU | | |
| AI3- | S | rArGrGmC*mC*fAmCfAdTfUmCmAmCmGmUmAmUmAmA | 25 | 73.37% |
| G2A | AS | mU*mU*fAmUfAmCfGmUfGmAfAmUfGmUfGmGfCmCfU*mG* | 26 | |
| | | mU | | |
| AI3- | S | rArGrGmC*mC*fAfCmAdTfUfCmAmCmGmUmAmUmAmA | 27 | 65.52% |
| G3A | AS | mU*mU*fAfUmAmCfGfUmGmAfAfUmGmUfGfGmCmCfU*mG* | 28 | |
| | | mU | | |
| AI3- | S | rArGrGmC*mC*mAfCfAdTmUmCmAmCmGmUmAmUmAmA | 29 | 58.10% |
| G4A | AS | mU*fU*fAmUmAfCfGmUmGfAfAmUmGfUfGmGmCfCmU*mG* | 30 | |
| | | mU | | |
| AI3- | S | rArGrGmC*fC*mAmCfAfUmUmCmAmCmGmUmAmUmAmA | 31 | 79.28% |
| G5A | AS | mU*mU*fAmUfAfCmGmUfGfAmAmUfGfUmGmGfCfCmU*mG* | 32 | |
| | | mU | | |
| AI3- | S | rArGrGmC*fC*fAmCmAfUfUmCmAmCmGmUmAmUmAmA | 33 | 86.25% |
| G6A | AS | mU*fU*mAfUfAmCmGfUfGmAmAfUfGmUmGfGfCmCmU*mG* | 34 | |
| | | mU | | |
| AI3- | S | rArGrGfC*mC*fAfCfAfUmUmCmAmCmGmUmAmUmAmA | 35 | 74.23% |
| G7A | AS | mU*fU*mAfUmAfCmGfUmGfAmAmUmGmUfGmGfCmCmU*m | 36 | |
| | | G*mU | | |
| AI3- | S | rArGrGmC*fC*mAfCfAfUfUmCmAmCmGmUmAmUmAmA | 37 | 39.32% |
| G8A | AS | mU*mU*fAmUfAmCfGmUfGmAmAmUmGfUmGfGmCfCmU*m | 38 | |
| | | G*mU | | |
| AI3- | S | rArGrGmC*mC*fAfCmAfUfUmCmAmCmGmUmAmUmAmA | 39 | 102.34% |
| G9A | AS | mU*fU*mAfUfAmCfGmUfGmAmAfUmGmUfGmGfCmCmU*mG | 40 | |
| | | *mU | | |
| AI3- | S | rArGrGmC*fC*fAfCfAfUmUmCmAmCmGmUmAmUmAmA | 41 | 96.37% |
| G10A | AS | mU*fU*mAfUmAfCmGfUmGfAmAmUmGmUmGfGmCfCmU*m | 42 | |
| | | G*mU | | |

S, sense; AS, antisense; m, 2′-O-methyl; f, 2′-fluoro; r, RNA residue with no modification;
d, DNA residue; * , phosphorothioate (PS) internucleotide linkages.

TABLE 2
Modified HIF-1A AT9 siRNAs and Activities Thereof for Inhibiting HIA-1A

| siRNA | | | | Expression |
|---|---|---|---|---|
| no. | Strand | Sense sequence (5′-3′) | Seq ID No. | level (%) |
| AT9 | S | UGAGGAAGUACCAUUAUAA | 43 | 36.35% |
| | AS | UUAUAAUGGUACUUCCUCAAU | 44 | |
| AT9- | S | mU*mG*mAmGfGmAfAmGfUmAfCmCmAmUmUmAmUmAmA | 45 | 29.73% |
| G1S | AS | mU*fU*mAfUmAfAmUfGmGfUmAfCmUfUmCfCmUfCmA*mA | 46 | |
| | | *mU | | |
| AT9- | S | mU*mG*mAmGmGfAmAfGdTfAmCmCmAmUmUmAmUmAmA | 47 | 90.54% |
| G2S | AS | mUmU*fAmUfAmAfUmGfGmUfAmCfUmUfCmCfUmCfA*mA | 48 | |
| | | *mU | | |
| AT9- | S | mU*mG*mAmGmGfAfAmGdTfAfCmCmAmUmUmAmUmAmA | 49 | 84.67% |
| G3S | AS | mU*mU*fAfUmAmAfUfGmGmUfAfCmUmUfCfCmUmCfA*mA | 50 | |
| | | *mU | | |
| AT9- | S | mU*mG*mAmGmGmAfAfGdTmAmCmCmAmUmUmAmUmAm | 51 | 43.13% |
| G4S | | A | | |
| | AS | mU*fU*fAmUmAfAfUmGmGfUfAmCmUfUfCmCmUfCmA*mA | 52 | |
| | | *mU | | |
| AT9- | S | mU*mG*mAmGfGmAmAfGfUmAmCmCmAmUmUmAmUmAm | 53 | 86.06% |
| G5S | | A | | |
| | AS | mU*mU*fAmUfAfAmUmGfGfUmAmCfUfUmCmCfUfCmA*mA | 54 | |
| | | *mU | | |
| AT9- | S | mUmG*mAmGfGfAmAmGfUfAmCmCmAmUmUmAmUmAmA | 55 | 66.97% |
| G6S | AS | mU*fU*mAfUfAmAmUfGfGmUmAfCfUmUmCfCfUmCmA*mA | 56 | |
| | | *mU | | |
| AT9- | S | mU*mG*mAfGmGfAfAfGfUmAmCmCmAmUmUmAmUmAmA | 57 | 80.66% |
| G7S | AS | mU*fU*mAfUmAfAmUfGmGfUmAmCmUmUfCmCfUmCmA*m | 58 | |
| | | A*mU | | |
| AT9- | S | mU*mG*mAmGfGmAfAfGfUfAmCmCmAmUmUmAmUmAmA | 59 | 39.96% |
| G8S | AS | mU*mU*fAmUfAmAfUmGfGmUmAmCmUfUmCfCmUfCmA*m | 60 | |
| | | A*mU | | |
| AT9- | S | mUmG*mAmGmGfAfAmGfUfAmCmCmAmUmUmAmUmAmA | 61 | 99.54% |
| G9S | AS | mU*fU*mAfUfAmAfUmGfGmUmAfCmUmUfCmCfUmCmA*m | 62 | |
| | | A*mU | | |
| AT9- | S | mU*mG*mAmGfGfAfAfGfUmAmCmCmAmUmUmAmUmAmA | 63 | 76.31% |
| G10S | AS | mU*fU*mAfUmAfAmUfGmGfUmAmCmUmUmCfCmUfCmA*m | 64 | |
| | | A*mU | | |
| AT9- | S | rUrGrAmG*fG*mAfAmGfUmAfCmCmAmUmUmAmUmAmA | 65 | 36.86% |
| GIA | AS | mU*fU*mAfUmAfAmUfGmGfUmAfCmUfUmCfCmUfCmA*mA | 66 | |
| | | *mU | | |
| AT9- | S | rUrGrAmG*mG*fAmAfGdTfAmCmCmAmUmUmAmUmAmA | 67 | 83.12% |
| G2A | AS | mUmU*fAmUfAmAfUmGfGmUfAmCfUmUfCmCfUmCfA*mA | 68 | |
| | | *mU | | |
| AT9- | S | rUrGrAmG*mG*fAfAmGdTfAfCmCmAmUmUmAmUmAmA | 69 | 93.95% |
| G3A | AS | mU*mU*fAfUmAmAfUfGmGmUfAfCmUmUfCfCmUmCfA*mA | 70 | |
| | | *mU | | |
| AT9- | S | rUrGrAmG*mG*mAfAfGdTmAmCmCmAmUmUmAmUmAmA | 71 | 48.07% |
| G4A | AS | mU*fU*fAmUmAfAfUmGmGfUfAmCmUfUfCmCmUfCmA*mA | 72 | |
| | | *mU | | |
| AT9- | S | rUrGrAmG*fG*mAmAfGfUmAmCmCmAmUmUmAmUmAmA | 73 | 78.46% |
| G5A | AS | mU*mU*fAmUfAfAmUmGfGfUmAmCfUfUmCmCfUfCmA*mA | 74 | |
| | | *mU | | |
| AT9- | S | rUrGrAmG*fG*fAmAmGfUfAmCmCmAmUmUmAmUmAmA | 75 | 82.74% |
| G6A | AS | mU*fU*mAfUfAmAmUfGfGmUmAfCfUmUmCfCfUmCmA*mA | 76 | |
| | | *mU | | |
| AT9- | S | rUrGrAfG*mG*fAfAfGfUmAmCmCmAmUmUmAmUmAmA | 77 | 78.10% |
| G7A | AS | mU*fU*mAfUmAfAmUfGmGfUmAmCmUmUfCmCfUmCmA*m | 78 | |
| | | A*mU | | |
| AT9- | S | rUrGrAmG*fG*mAfAfGfUfAmCmCmAmUmUmAmUmAmA | 79 | 64.47% |
| G8A | AS | mU*mU*fAmUfAmAfUmGfGmUmAmCmUfUmCfCmUfCmA*m | 80 | |
| | | A*mU | | |
| AT9- | S | rUrGrAmG*mG*fAfAmGfUfAmCmCmAmUmUmAmUmAmA | 81 | 87.86% |
| G9A | AS | mU*fU*mAfUfAmAfUmGfGmUmAfCmUmUfCmCfUmCmA*m | 82 | |
| | | A*mU | | |
| AT9- | S | rUrGrAmG*fG*fAfAfGfUmAmCmCmAmUmUmAmUmAmA | 83 | 80.48% |
| G10A | AS | mU*fU*mAfUmAfAmUfGmGfUmAmCmUmUmCfCmUfCmA*m | 84 | |
| | | A*mU | | |

S, sense;
AS, antisense;
m, 2′-O-methyl;
f, 2′-fluoro;
r, unmodified nucleotides;
d, deoxyribonucleotide;
*, phosphorothioate (PS) internucleotide linkages.

20 modified siRNAs for each of AI3 and AT9 as listed in Tables 1 and Table 2 were investigated for their activity in inhibiting target gene expression. Each of the modified siRNAs, as well as the unmodified control, was transfected into human hepatocellular carcinoma (HepG2) cells at a concentration of 10 nM. At 24 hr post-transfection, HIF-1A mRNA levels in the transfected cells were detected via RT-qPCR. The HIF-1A mRNA expression levels (%) in transfected cells were calculated using the HIF-1A mRNA level in non-transfected cells as 100%. The results are provided in Tables 1 and 2 above.

A brief description of the assay methods is provided below:

### Culture of HepG2 Cells

The human hepatocellular carcinoma (HepG2) cell line was maintained in minimum essential medium (Gibco, ThermoFisher Scientific, USA) containing 10% fetal bovine serum (Gibco, ThermoFisher Scientific), 200 units/mL penicillin plus 200 units/mL streptomycin at 37° C. with 5% CO2.

siRNA Transfection in HepG2 Cells

HepG2 cells were seeded at 5×10⁵ cells/well in 24-well culture plates. After 18 hr incubation, the medium was replaced with 500 ul of fresh growth medium. The complex composition of siRNA and RNAiMax for each well was prepared as following: (1) 1 ul of siRNA was added to 50 ul of Opti-MEM; (2) 1.5 ul of RNAiMax was added to 50 ul of Opti-MEM; (3) Gently mix (1) and (2) and incubate at room temperature for 10 minutes. Transfection was carried out by adding 100 ul of siRNA/RNAiMax complex to each well. Cells were then incubated for 24 hr prior to RNA purification.

### RT-qPCR

Total RNA was extracted with an RNeasy kit (Qiagen) according to the manufacturer's protocol. HIF1A mRNA levels was quantified using one-step real-time quantitative PCR with iTaq Universal Probes one-step kit (Bio-Rad), performed on the LightCycler 480 (Roche Diagnostics). RT-qPCR was performed in triplicates with 50 ng of total RNA, 500 nM each of forward, reverse primer and probe, 0.25 ul of reverse transcriptase, and 2×iTaq Master Mix in a total volume of 10 ul in a 384-well plate. Primers and Probes for HIF1A and GAPDH (Table 4) were synthesized from Integrated DNA Technologies. The cycling condition was in accordance with the manufacturer's recommended cycling parameters: 50° C. for 10 min, 95° C. for 2 min, and 40 cycles of 95° C. for 15 s and 60° C. for 1 min. Gene expression fold change was calculated using the ΔΔCt method. HIF1A mRNA was normalized to constitutively expressed GAPDH mRNA.

More modification patterns for AI3 and AT9 siRNAs were further designed and screened in HepG2 cells as listed in Table 3.

TABLE 3
Modified HIFIA AI3 and AT9 siRNAs and Inhibitory Activities Thereof

| | | | | HIF1A |
|---|---|---|---|---|
| siRNA | | | Seq | expression |
| No. | Strand | Sequence (5′-3′) | ID No. | level (%) |
| AI3 | S | AGGCCACAUUCACGUAUAA | 1 | 58.24% |
| | AS | UUAUACGUGAAUGUGGCCUGU | 2 | |
| AI3-G4S | S | mA*mG*mGmCmCmAfCfAdTmUmCmAmCmGmUmAmUmA | 9 | 49.65% |
| | | mA | | |
| | AS | mU*fU*fAmUmAfCfGmUmGfAfAmUmGfUfGmGmCfCmU*m | 10 | |
| | | G*mU | | |
| AI3-G8A | S | rArGrGmC*fC*mAfCfAfUfUmCmAmCmGmUmAmUmAmA | 37 | 55.10% |
| | AS | mU*mU*fAmUfAmCfGmUfGmAmAmUmGfUmGfGmCfCmU* | 38 | |
| | | mG*mU | | |
| AI3-G12S | S | mA*mG*mGmCmCmAfCfAfUmUmCmAmCmGmUmAmUmA | 85 | 65.82% |
| | | mA | | |
| | AS | mU*fU*fAmUmAfCfGmUmGfAfAmUmGfUfGmGmCfCmU*m | 86 | |
| | | G*mU | | |
| AI3-G15S | S | mA*mG*fGmCmCmAfCfAdTmUmCmAmCmGmUmAmUmA | 87 | 98.17% |
| | | mA | | |
| | AS | mU*mU*fAmUmAfCfGmUmGfAfAmUmGfUfGmGmCfCmU* | 88 | |
| | | mG*mU | | |
| AI3-G16S | S | mA*mG*mGmCmCmAfCfAdTfUmCmAmCmGmUmAmUmA | 89 | 62.42% |
| | | mA | | |
| | AS | mU*fU*fAmUmAfCfGmUfGmAfAmUmGfUfGmGmCfCmU*m | 90 | |
| | | G*mU | | |
| AI3-G17S | S | mA*mG*mGmCmCmAfCfAdTmUmCmAmCmGmUmAmUmA | 91 | 65.22% |
| | | mA | | |
| | AS | mU*fU*mAmUfAmCfGfUmGfAfAmUmGfUfGmGmCfCmU*m | 92 | |
| | | G*mU | | |
| AI3-G18S | S | mA*mG*mGmCmCmAfCfAdTfUmCmAmCmGmUmAmUmA | 93 | 78.82% |
| | | mA | | |
| | AS | mU*fU*mAfUfAmCmGfUfGmAfAmUmGfUfGmGmCfCmU*m | 94 | |
| | | G*mU | | |
| AI3-G19S | S | mA*mG*mGmCmCmAfCfAdTfUmCmAmCmGmUmAmUmA | 95 | 67.83% |
| | | mA | | |
| | AS | mU*fU*mAmUfAmCmGfUfGmAfAmUmGfUfGmGmCfCmU* | 96 | |
| | | mG*mU | | |
| AI3-G22A | S | rArGrGmC*fC*mAfCfAfUfUmCmAmCmGmUmAmUmAmA | 97 | 80.29% |
| | AS | mU*fU*mAfUmAfCmGfUfGmAmAmUmGfUmGfGmCfCmU* | 98 | |
| | | mG*mU | | |
| AI3-G24S | S | mA*mG*mGmCfCmAfCfAfUfUmCmAmCmGmUmAmUmAm | 99 | 87.26% |
| | | A | | |
| | AS | mU*mU*fAmUfAmCmGfUfGmAmAmUmGfUmGmGfCfCmU* | 100 | |
| | | mG*mU | | |
| AI3-G25S | S | mA*mG*mGmCfCmAfCfAfUfUmCmAmCmGmUmAmUmAm | 101 | 50.93% |
| | | A | | |
| | AS | mU*fU*mAfUfAmCmGfUfGmAmAmUmGfUmGfGmCfCmU* | 102 | |
| | | mG*mU | | |
| AI3-G26S | S | mA*mG*fGmCmCmAfCfAfUfUmCmAmCmGmUmAmUmAm | 103 | 72.03% |
| | | A | | |
| | AS | mU*mU*fAmUfAmCfGmUfGmAmAmUmGfUfGmGmCfCmU* | 104 | |
| | | mG*mU | | |
| AI3-G29S | S | mA*mG*mGfCmCmAfCfAdTfUmCmAmCmGmUmAmUmAm | 105 | 88.27% |
| | | A | | |
| | AS | mU*fU*mAfUmAfCmGfUfGmAfAmUmGfUmGmGfCmCmU* | 106 | |
| | | mG*mU | | |
| AT9-G1S | S | mU*mG*mAmGfGmAfAmGfUmAfCmCmAmUmUmAmUmA | 45 | 55.86% |
| | | mA | | |
| | AS | mU*fU*mAfUmAfAmUfGmGfUmAfCmUfUmCfCmUfCmA*m | 46 | |
| | | A*mU | | |
| AT9-G1A | S | rUrGrAmG*fG*mAfAmGfUmAfCmCmAmUmUmAmUmAmA | 65 | 52.24% |
| | AS | mU*fU*mAfUmAfAmUfGmGfUmAfCmUfUmCfCmUfCmA*m | 66 | |
| | | A*mU | | |
| AT9- | S | rUrGrAmG*fG*mAfAmGfUmAfCmCmAmUmUmAmUmAmA | 107 | 53.71% |
| G31A | AS | mU*fU*mAfUmAfAmUfGmGfUmAfCmUfUmCfCmUmCmA* | 108 | |
| | | mA*mU | | |
| AT9- | S | rUrGrAmG*fG*mAfAmGfUmAfCmCmAmUmUmAmUmAmA | 109 | 61.27% |
| G32A | AS | mU*mU*mAfUmAfAmUfGmGfUmAfCmUfUmCfCmUmCmA* | 110 | |
| | | mA*mU | | |

S, sense;
AS, antisense;
m, 2′-O-methyl;
f, 2′-fluoro;
r, unmodified nucleotides;
d, deoxyribonucleotide;
*, phosphorothioate (PS) internucleotide linkages.

TABLE 4
Primers and Probes for Use in RT-qPCR to Detect
the Relative Expression Levels of HIF1A

| Gene | Primer/Probe | Sequence |
|---|---|---|
| HIFIA | HIFIA_F | 5′-CTCTGATCATCTGACCAAAACTCA-3′ |
| | | (SEQ ID NO: 137) |
| | HIFIA_R | 5′-CAACCCAGACATATCCACCTC-3′ |
| | | (SEQ ID NO: 138) |
| | HIFIA_Probe | 5′-/56FAM/TGGCAAGCA/ZEN/TCCTGTA |
| | | CTGTCCTG/3IABKFQ/-3′ |
| | | (SEQ ID NO: 139) |
| GAPDH | GAPDH_F | 5′-ACATCGCTCAGACACCATG-3′ |
| | | (SEQ ID NO: 140) |
| | GAPDH_R | 5′-TGTAGTTGAGGTCAATGAAGGG-3′ |
| | | (SEQ ID NO: 141) |
| | GAPDH_Probe | 5′-56FAM/AAGGTCGGA/ZEN/GTCAACG |
| | | GATTTGGTC/3IABKFQ/-3′ |
| | | (SEQ ID NO: 142) |

Table 3 presents the corresponding HIF1A expression levels in cells transfected with each of the identified siRNAs at a concentration of 0.5 nM. Four modification patterns for the anti-HIF-1A AI3 siRNA were identified from the in vitro screening studies of chemical modifications as having similar or higher interference efficiencies relative to unmodified AI3. Modified AI3 siRNAs with these modification patterns include AI3-G4S, AI3-G8S, AI3-G8A and AI3-G25S. Similarly, four modified AT9 siRNAs were identified from the in vitro screening assay as having similar interference efficiency relative to the unmodified AT9. Examples include AT9-G1S, AT9-G1A, AT9-G8S, and AT9-G31A.

### Example 2: Chemically Modified siRNAs Targeting SARS-COV-2 and Interference Efficiencies Thereof

To further identify and validate additional chemical modification patterns for siRNAs with desirable biological features, two siRNAs targeting the genomic RNA and subgenomic mRNAs of the SARS-COV-2 virus, C6 and C8, were modified in patterns listed in Table 5.

TABLE 5
Modified SARS-COV-2 siRNAs and Inhibitory Activities Thereof

| siRNA | | | | Expression |
|---|---|---|---|---|
| No. | Strand | Sequence (5′-3′) | Seq ID no. | level (%) |
| C6 | S | CUGUCAAACCCGGUAAUUU | 111 | 16.50% |
| | AS | AAAUUACCGGGUUUGACAGUU | 112 | |
| C6-G4S | S | mC*mU*mGmUmCmAfAfAdCmCmCmGmGmUmAmA | 113 | 100.00% |
| | | mUmUmU | | |
| | AS | mA*fA*fAmUmUfAfCmCmGfGfGmUmUfUfGmAmCfA | 114 | |
| | | mG*mU*mU | | |
| C6-G8S | S | mC*mU*mGmUfCmAfAfAfCfCmCmGmGmUmAmAmU | 115 | 59.30% |
| | | mUmU | | |
| | AS | mA*mA*fAmUfUmAfCmCfGmGmGmUmUfUmGfAmCf | 116 | |
| | | AmG*mU*mU | | |
| C6-G8A | S | rCrUrGmU*fC*mAfAfAfCfCmCmGmGmUmAmAmUm | 117 | 4.90% |
| | | UmU | | |
| | AS | mA*mA*fAmUfUmAfCmCfGmGmGmUmUfUmGfAmCf | 118 | |
| | | AmG*mU*mU | | |
| C6- | S | rCrUrGmU*fC*mAfAfAfCfCmCmGmGmUmAmAmUm | 119 | 6.50% |
| G22A | | UmU | | |
| | AS | mA*fA*mAfUmUfAmCfCfGmGmGmUmUfUmGfAmCf | 120 | |
| | | AmG*mU*mU | | |
| C6-G25S | S | mC*mU*mGmUfCmAfAfAfCfCmCmGmGmUmAmAmU | 121 | 13.70% |
| | | mUmU | | |
| | AS | mA*fA*mAfUfUmAmCfCfGmGmGmUmUfUmGfAmCf | 122 | |
| | | AmG*mU*mU | | |
| C6-G1S | S | mC*mU*mGmUfCmAfAmAfCmCfCmGmGmUmAmAm | 123 | 20.10% |
| | | UmUmU | | |
| | AS | mA*fA*mAfUmUfAmCfCmGfGmGfUmUfUmGfAmCfA | 124 | |
| | | mG*mU*mU | | |
| C6-GIA | S | rCrUrGmU*fC*mAfAmAfCmCfCmGmGmUmAmAmUm | 125 | 6% |
| | | UmU | | |
| | AS | mA*fA*mAfUmUfAmCfCmGfGmGfUmUfUmGfAmCfA | 126 | |
| | | mG*mU*mU | | |
| C6- | S | rCrUrGmU*fC*mAfAmAfCmCfCmGmGmUmAmAmUm | 127 | 14.10% |
| G31A | | Uml | | |
| | AS | mA*fA*mAfUmUfAmCfCmGfGmGfUmUfUmGfAmCm | 128 | |
| | | AmG*mU*mU | | |
| C8 | S | GCCACUAGUCUCUAGUCAA | 129 | 2% |
| | AS | UUGACUAGAGACUAGUGGCUU | 130 | |
| C8-G25S | S | mG*mC*mCmAfCmUfAfGfUfCmUmCmUmAmGmUmC | 131 | 12% |
| | | mAmA | | |
| | AS | mU*fU*mGfAfCmUmAfGfAmGmAmCmUfAmGfUmGf | 132 | |
| | | GmC*mU*mU | | |

S, sense;
AS, antisense;
m, 2′-O-methyl;
f, 2′-fluoro;
r, unmodified nucleotides;
d, deoxyribonucleotide;
*, phosphorothioate (PS) internucleotide linkages

Each of the modified siRNAs targeting SARS-COV-2, as well as the unmodified counterpart, was transfected into Vero E6 cells. Briefly, the Vero cells were reverse-transfected with siRNA candidates and then seeded into 24-well culture plates. At 24 hr post-transfection, the siRNA-transfected cells were infected by SARS-COV-2 virus with an MOI of 0.1. After 1 h incubation, the inoculum was removed and cells were washed with phosphate-buffered saline (PBS). Fresh medium was added and incubation was continued for 24 h. After 24 h incubation, the knockdown of SARS-CoV-2 RNA genome by siRNAs in Vero E6 cells was determined by RT-qPCR targeting the E (envelope) gene.

Total cellular RNA was extracted with a NucleoSpin RNA mini kit (Macherey-Nagel, Düren, Germany). The amount of viral RNA was determined by reverse transcription-quantitative polymerase chain reaction (RT-qPCR) of viral E gene on a QuantStudio 5 Real-Time PCR System (Applied Biosystems) using an iTaq Universal Probes One-Step RT-PCR Kit (Bio-Rad). The primers and probe targeting the SARS-COV-2 were as follows: forward primer, 5′-ACAGGTACGTTAATAGTTAATAGCGT-3′ (SEQ ID NO:143); reverse primer, 5′-ACATTGCAGCAGTACGCACACA-3′ (SEQ ID NO: 144); and probe, 5′-ACACTAGCCATCCTTACTGCGCTTCG-3′ (SEQ ID NO: 145). Plasmid containing partial E fragment was used as a standard to calculate the viral load (copies/μl).

At a concentration of 20 nM for each siRNA, five modification patterns were identified for C6 and C8 siRNA that maintain good silencing efficiency as compared with unmodified siRNA. The following modified siRNAs include the five modification patterns: C6-G8A, C6-G22A, C6-G25S, 6-G1A and C6-G31A.

Phosphorothioate (PS) internucleotide linkages at terminal ends of siRNAs to protect siRNA against nuclease degradation does not affect the interference efficiency of the siRNA as shown in Table 6.

TABLE 6
Validation of Interference Efficiencies for Modified C6 and C8 siRNAs.

| siRNA | | | Seq | Expression |
|---|---|---|---|---|
| No. | Strand | Sequence (5′-3′) | ID No. | level (%) |
| C6 | S | mC*mU*mGmUfCmAfAfAfCfCmCmGmGmUmAmAmUmUmU | 121 | 3.60% |
| G25S | AS | mA*fA*mAfUfUmAmCfCfGmGmGmUmUfUmGfAmCfAmG*m | 122 | |
| | | U*mU | | |
| C6- | S | mC*mU*mGmUfCmAfAfAfCfCmCmGmGmUmAmAmU*mU* | 133 | 1.00% |
| G25S-2 | | mU | | |
| | AS | mA*fA*mAfUfUmAmCfCfGmGmGmUmUfUmGfAmCfAmG*m | 134 | |
| | | U*mU | | |
| C8- | S | mG*mC*mCmAfCmUfAfGfUfCmUmCmUmAmGmUmCmAmA | 131 | 18.60% |
| G25S | AS | mU*fU*mGfAfCmUmAfGfAmGmAmCmUfAmGfUmGfGmC*m | 132 | |
| | | U*mU | | |
| C8- | S | mG*mC*mCmAfCmUfAfGfUfCmUmCmUmAmGmUmC*mA* | 135 | 20.00% |
| G25S-2 | | mA | | |
| | AS | mU*fU*mGfAfCmUmAfGfAmGmAmCmUfAmGfUmGfGmC*m | 136 | |
| | | U*mU | | |

S, sense;
AS, antisense;
m, 2′-O-methyl;
f, 2′-fluoro;
r, unmodified nucleotides;
d, deoxyribonucleotide;
*, phosphorothioate (PS) internucleotide linkages.

### Example 3: Chemically Modified siRNAs with 5′-Phosphate Modifications Targeting HBx Transcript and Interference Efficiencies Thereof

To investigate whether 5′ phosphate modifications can improve the interference efficiencies of siRNAs having the chemical modification patterns as disclosed herein, the 5′ end of antisense strand of exemplary siRNAs targeting a highly conserved sequence in the X open reading frame (HBx) of all the hepatitis B virus transcripts were further modified with E-vinylphosphonate or phosphorothioate. The sequences and modification of the exemplary anti-HBx siRNAs are shown in Table 7. Chemical structures of the 5′-phosphate modifications E-vinylphosphonate and phosphorothioate are shown in FIG. 2A.

TABLE 7
Modified siRNAs Targeting HBx

| SIRNA | | | Seq |
|---|---|---|---|
| No. | Strand | Sequence (5′-3′) | ID No. |
| X32- | S | mG*mG*mAmGfGmCfUfGfUfAmGmGmCmAmUmAmAmAmA | 146 |
| G25S- | AS | mU*fU*mUfUfA*mU*mGfCfCmUmAmCmAfGmCfCmUfCmC* | 147 |
| PS6 | | mU*mU | |
| X32- | S | mG*mG*mAmGfGmCfUfGfUfAmGmGmCmAmUmAmAmAmA | 148 |
| G25S- | AS | 5′-E-VP mU*fU*mUfUfA*mU*mGfCfCmUmAmCmAfGmCfC | 149 |
| PS6- | | mUfCmC*mU*mU | |
| VP | | | |
| X32- | S | mG*mG*mAmGfGmCfUfGfUfAmGmGmCmAmUmAmAmAmA | 150 |
| G25S- | AS | P*mU*fU*mUfUfA*mU*mGfCfCmUmAmCmAfGmCfCmUfCm | 151 |
| PS6-5S | | C*mU*mU | |

S, sense;
AS, antisense;
m, 2′-O-methyl;
f, 2′-fluoro;
5′-E-VP, 5′-E- vynylphosphonate
P*, 5′-phosphorothioate
*, phosphorothioate (PS) internucleotide linkages.

The human hepatocellular carcinoma cell line Hep3B was maintained in DMEM medium (Gibco, ThermoFisher Scientific, USA) containing 10% fetal bovine serum (Gibco, ThermoFisher Scientific) at 37° C. with 5% CO₂.

The Hep3B cells were seeded at 4×10⁴ cells/well in 24-well culture plates. After 18-hr incubation, the Hep3B cells were transfected with each of the modified anti-HBx siRNA following the same approach as previously described in connection with HepG2 cells. See Example 2 above. Cells were then incubated for 24-hr and the total RNAs were extracted from siRNA-transfected Hep3B cells using a RNeasy kit (Qiagen). Reverse transcription reactions were performed using a Maxima First-Strand cDNA Synthesis kit (Thermo Fisher Scientific) and 100 ng of the total cellular RNAs extracted. qPCR was carried out on a LightCycler 480 using SYBR Green I Master (Roche Diagnostics) with 1:5 dilutions of cDNA. The primer sets used to detect HBx transcripts are shown in Table 8. Each sample was assayed in triplicates to determine an average threshold cycle (Ct) value. Gene expression fold change was calculated using the ΔΔCt method. The mRNA level of each gene was normalized to constitutively expressed GAPDH mRNAs, which are used as a housekeeping control.

TABLE 8
RT-qPCR Primers for the Quantification
of HBx Expression.

| Gene | Primer | Sequence |
|---|---|---|
| HBx | HBx F | 5′-CCGTCTGTGCCTTCTCATCTGC-3′ |
| | | (SEQ ID NO: 152) |
| | HBx R | 5′-ACCAATTTATGCCTACAGCCTCC-3′ |
| | | (SEQ ID NO: 153) |
| GAPDH | GAPDH F | 5′-CATCACTGCCACCCAGAAGA-3′ |
| | | (SEQ ID NO: 154) |
| | GAPDH R | 5′-CCTGCTTCACCACCTTCTTG-3′ |
| | | (SEQ ID NO: 155) |

The human hepatocellular carcinoma cell line Hep3 B cells contains an integrated hepatitis B virus genome and thus is capable of expressing the HBx transcripts. The anti-HBx siRNAs with or without 5′ phosphate modifications (10 or 1.2 nM siRNA) were transfected into Hep3B cells for 24 hours and the expression of HBx transcripts was quantified by RT-qPCR. As shown in FIG. 2B, the HBx siRNAs with E-vinylphosphonate (X32-G25S-PS6-VP) or phosphorothioate (X32-G25S-PS6-5S) 5′-phosphate modification improved the siRNA interference efficiency to reduce expression of the HBx transcripts, as compared with the HBx siRNA counterpart without the 5′-phosphate modification (X32-G25S-PS6).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

The following listing of claims replaces any previous listing of claims:

1. A modified small interfering RNA (siRNA), comprising a sense strand having 19 nucleotides and an antisense strand having 21 nucleotides,
wherein (a) the antisense strand contains (i) 7 to 9 2′-fluoro-modified nucleotides, and (ii) 12 to 14 2′-O-methyl-modified nucleotides; and (b) the sense strand contains (i) 2 to 5 2′-fluoro-modified nucleotides, and (ii) 11 to 16 2′-O-methyl-modified nucleotides;
wherein the sense strand, the antisense strand, or both contain one or more phosphothioate (PS) bonds; and
wherein the sense strand and the antisense stand form a double-stranded siRNA, optionally with a two-nucleotide overhang at the 3′ end of the antisense strand.

2. The modified siRNA of claim 1, wherein the antisense strand contains a modified 5′-phosphate.

3. The modified siRNA of claim 2, wherein the modified 5′-phosphate is E-vinyl phosphonate or phosphorothiorate.

4. The modified siRNA of claim 1, wherein the antisense strand contains the 2′-fluoro-modified nucleotides at 7 or more of positions 2-12, 14-16, and 18.

5. The modified siRNA of claim 4, wherein the antisense strand comprises 2′-fluoro-modified nucleotides at position 2 or 3, and position 14, and optionally position 18.

6. The modified siRNA of claim 1, wherein in (a), the antisense strand contains the 2′-O-methyl-modified nucleotides at 12 or more of positions 1, 2-13, and 15-21.

7. The modified siRNA of claim 6, wherein the antisense strand comprises 2′-O-methyl-modified nucleotides at positions 1, 13, 17 and 19-21.

8. The modified siRNA of claim 1, wherein in (b), the sense strand contains the 2′-fluoro-modified nucleotides at two or more of positions 5 and 7-11.

9. The modified siRNA of claim 8, wherein the sense strand comprises 2′-fluoro-modified nucleotides at positions 5 and 7-10 or at positions 5, 7, 9, and 11.

10. The modified siRNA of claim 1, wherein the sense strand contains the 2′-O-methyl-modified nucleotides at 11 or more of positions of 1-6, 8, and 10-19.

11. The modified siRNA of claim 10, wherein the sense strand comprises 2′-O-methyl-modified nucleotides at positions 4, 6, and 11-19.

12. The modified siRNA of claim 11, wherein the sense strand further comprises 2′-O-methyl-modified nucleotides at positions 8 and 10.

13. The modified siRNA of claim 10, wherein one or more positions 1-3 in the sense strand contain 2′-O-methyl-modified nucleotides.

14. The modified siRNA of claim 10, wherein one or more of positions 1-3 in the sense strand are not modified.

15. The modified siRNA of claim 1, wherein the sense strand contains a 2′-deoxynucleotide, which optionally is located as position 9.

16. The modified siRNA of claim 1, wherein the antisense strand contains PS bonds between positions 1 and 2, positions 2 and 3, positions 19 and 20, and/or positions 20 and 21.

17. The modified siRNA of claim 1, wherein the sense strand contains PS bonds between positions 1 and 2, positions 2 and 3, positions 4 and 5, positions 5 and 6, positions 17 and 18, and/or positions 18 and 19.

18. The modified siRNA of claim 1, wherein the antisense strand of (a) and the sense strand of (b) have the following formulas:
(i)
5′-mffmmffmmffmmffmmfmmm-3′
3′-mmmmmmmmmmdffmmmmmm-5′;
(ii)
5′-mmfmfmfmfmmmmfmfmfmmm-3′
3′-mmmmmmmmmffffmfmmmm-5′;
(iii)
5′-mmfmfmfmfmmmmfmfmfmmm-3′
3′-mmmmmmmmmffffmfmrrr-5′;
(iv)
5′-mfmfmfmffmmmmfmfmfmmm-3′
3′-mmmmmmmmmffffmfmrrr-5′
(v)
5′-mfmffmmffmmmmfmfmfmmm-3′
3′-mmmmmmmmmffffmfmmmm-5′;
(vi)
5′ -mfmfmfmfmfmfmfmfmfmmm-3′
3′-mmmmmmmmfmfmfmfmmmm-5′;
(vii)
5′-mfmfmfmfmfmfmfmfmfmmm-3′
3′-mmmmmmmmfmfmfmfmrrr-5″ or
(viii)
5′-mfmfmfmfmfmfmfmfmmmmm-3′
3′-mmmmmmmmfmfmfmfmrrr-3′; and
wherein m represents 2′-O-methyl nucleotide, f represents 2′-fluoro nucleotide, r represents unmodified nucleotide, and d represents DNA residue.

19. The modified siRNA of claim 18, wherein the antisense strand of (a) and the sense strand of (b) have the following formulas:
(i)
5′-m*f*fmmffmmffmmffmmfm*m*m-3′
3′-m*m*mmmmmmmmdffmmmm*m*m-5′;
(ii)
5′-m*m*fmfmfmfmmmmfmfmfm*m*m-3′
3′-m*m*mmmmmmmffffmfmm*m*m-5′;
(iii)
5′-m*m*fmfmfmfmmmmfmfmfm*m*m-3′
3′-m*m*mmmmmmmffffm*f*mrrr-5′;
(iv)
5′-m*f*mfmfmffmmmmfmfmfm*m*m-3′
3′-m*m*mmmmmmmffffm*f*mrrr-5′
(v)
5′-m*f*mffmmffmmmmfmfmfm*m*m-3′
3′-m*m*mmmmmmmffffmfmm*m*m-5′;
(vi)
5′-m*f*mfmfmfmfmfmfmfmfm*m*m-3′
3′-m*m*mmmmmmfmfmfmfmm*m*m-5′;
(vii)
5′-m*f*mfmfmfmfmfmfmfmfm*m*m-3′
3′-m*m*mmmmmmfmfmfm*f*mrrr-5″ or
(viii)
5′-m*f*mfmfmfmfmfmfmfmmm*m*m-3′
3′-m*m*mmmmmmfmfmfm*f*mrrr-3′; and
wherein m represents 2′-O-methyl nucleotides, f represents 2′-fluoro nucleotides, r represents unmodified nucleotides, d represents DNA residue, and * represents PS bonds.

20. The modified siRNA of claim 1, wherein the sense strand is conjugated to a targeting moiety, which optionally is N-Acetylgalactosamine (GalNAc).

21. A pharmaceutical composition comprising the siRNA of claim 1 and a pharmaceutically acceptable carrier.

22. A method for modulating gene expression, comprising contacting cells with the siRNA of claim 1, or a pharmaceutical composition comprising the siRNA and a pharmaceutically acceptable carrier, wherein the siRNA targets a gene of interest.

23. The method of claim 22, wherein the contacting step is performed by administering the siRNA or the pharmaceutical composition to a subject having a disease associated with the gene of interest.


SEQUENCE LISTING (155 sequences)

SEQ ID NO: 1 - RNA, 19 nt, synthetic construct
  source 1..19: mol_type = other RNA
aggccacattcacgtataa

SEQ ID NO: 2 - RNA, 21 nt, synthetic construct
  source 1..21: mol_type = other RNA
ttatacgtgaatgtggcctgt

SEQ ID NO: 3 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
aggccacattcacgtataa

SEQ ID NO: 4 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 5 - DNA, 19 bp, synthetic construct
  modified_base 1..3: mod_base = OTHER; note = modified with phosphorothioate linker
  modified_base 1..5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 6 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12: mod_base = OTHER; note = modified with 2'-O-methyl
  modified_base 13: mod_base = OTHER; note = modified with 2'-Fluoro
  modified_base 14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttatacgtgaatgtggcctgt

SEQ ID NO: 7 - DNA, 19 bp, synthetic construct
  modified_base 1..3: mod_base = OTHER; note = modified with phosphorothioate linker
  modified_base 1..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 10..11: mod_base = OTHER; note = modified with 2'-Fluoro
  modified_base 12..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 8 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3..4: mod_base = OTHER; note = modified with 2'-Fluoro
  modified_base 5..6: mod_base = OTHER; note = modified with 2'-O-methyl
  modified_base 7..8: mod_base = OTHER; note = modified with 2'-Fluoro
  modified_base 9..10: mod_base = OTHER; note = modified with 2'-O-methyl
  modified_base 11..12: mod_base = OTHER; note = modified with 2'-Fluoro
  modified_base 13..14: mod_base = OTHER; note = modified with 2'-O-methyl
  modified_base 15..16: mod_base = OTHER; note = modified with 2'-Fluoro
  modified_base 17..18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttatacgtgaatgtggcctgt

SEQ ID NO: 9 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 10 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 11 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 12 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..12: mod_base = OTHER; note = modified with 2'-O-methyl
  modified_base 13..14: mod_base = OTHER; note = modified with 2'-Fluoro
  modified_base 15..16: mod_base = OTHER; note = modified with 2'-O-methyl
  modified_base 17..18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 13 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 14 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..11: mod_base = OTHER; note = modified with 2'-O-methyl
  modified_base 12..13: mod_base = OTHER; note = modified with 2'-Fluoro
  modified_base 14..15: mod_base = OTHER; note = modified with 2'-O-methyl
  modified_base 16..17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 15 - RNA, 19 nt, synthetic construct
  modified_base 1..3: mod_base = OTHER; note = modified with phosphorothioate linker
  modified_base 1..3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 16 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 17 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 18 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 19 - RNA, 19 nt, synthetic construct
  modified_base 1..3: mod_base = OTHER; note = modified with phosphorothioate linker
  modified_base 1..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 20 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..11: mod_base = OTHER; note = modified with 2'-O-methyl
  modified_base 12: mod_base = OTHER; note = modified with 2'-Fluoro
  modified_base 13..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 21 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 22 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 23 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
aggccacattcacgtataa

SEQ ID NO: 24 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 25 - DNA, 19 bp, synthetic construct
  modified_base 4..6: mod_base = OTHER; note = modified with phosphorothioate linker
  modified_base 4..5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 26 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12: mod_base = OTHER; note = modified with 2'-O-methyl
  modified_base 13: mod_base = OTHER; note = modified with 2'-Fluoro
  modified_base 14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttatacgtgaatgtggcctgt

SEQ ID NO: 27 - DNA, 19 bp, synthetic construct
  modified_base 4..6: mod_base = OTHER; note = modified with phosphorothioate linker
  modified_base 4..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 10..11: mod_base = OTHER; note = modified with 2'-Fluoro
  modified_base 12..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 28 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3..4: mod_base = OTHER; note = modified with 2'-Fluoro
  modified_base 5..6: mod_base = OTHER; note = modified with 2'-O-methyl
  modified_base 7..8: mod_base = OTHER; note = modified with 2'-Fluoro
  modified_base 9..10: mod_base = OTHER; note = modified with 2'-O-methyl
  modified_base 11..12: mod_base = OTHER; note = modified with 2'-Fluoro
  modified_base 13..14: mod_base = OTHER; note = modified with 2'-O-methyl
  modified_base 15..16: mod_base = OTHER; note = modified with 2'-Fluoro
  modified_base 17..18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttatacgtgaatgtggcctgt

SEQ ID NO: 29 - DNA, 19 bp, synthetic construct
  modified_base 4..6: mod_base = OTHER; note = modified with phosphorothioate linker
  modified_base 4..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 30 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 31 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 32 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..12: mod_base = OTHER; note = modified with 2'-O-methyl
  modified_base 13..14: mod_base = OTHER; note = modified with 2'-Fluoro
  modified_base 15..16: mod_base = OTHER; note = modified with 2'-O-methyl
  modified_base 17..18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 33 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 34 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..11: mod_base = OTHER; note = modified with 2'-O-methyl
  modified_base 12..13: mod_base = OTHER; note = modified with 2'-Fluoro
  modified_base 14..15: mod_base = OTHER; note = modified with 2'-O-methyl
  modified_base 16..17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 35 - RNA, 19 nt, synthetic construct
  modified_base 4..6: mod_base = OTHER; note = modified with phosphorothioate linker
  modified_base 4: mod_base = OTHER; note = modified with 2'-Fluoro
  modified_base 5: mod_base = OTHER; note = modified with 2'-O-methyl
  modified_base 6..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 36 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 37 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 38 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 39 - RNA, 19 nt, synthetic construct
  modified_base 4..6: mod_base = OTHER; note = modified with phosphorothioate linker
  modified_base 4..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 40 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..11: mod_base = OTHER; note = modified with 2'-O-methyl
  modified_base 12: mod_base = OTHER; note = modified with 2'-Fluoro
  modified_base 13..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 41 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 42 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 43 - RNA, 19 nt, synthetic construct
  source 1..19: mol_type = other RNA
tgaggaagtaccattataa

SEQ ID NO: 44 - RNA, 21 nt, synthetic construct
  source 1..21: mol_type = other RNA
ttataatggtacttcctcaat

SEQ ID NO: 45 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
tgaggaagtaccattataa

SEQ ID NO: 46 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 47 - DNA, 19 bp, synthetic construct
  modified_base 1..3: mod_base = OTHER; note = modified with phosphorothioate linker
  modified_base 1..5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 48 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12: mod_base = OTHER; note = modified with 2'-O-methyl
  modified_base 13: mod_base = OTHER; note = modified with 2'-Fluoro
  modified_base 14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttataatggtacttcctcaat

SEQ ID NO: 49 - DNA, 19 bp, synthetic construct
  modified_base 1..3: mod_base = OTHER; note = modified with phosphorothioate linker
  modified_base 1..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 10..11: mod_base = OTHER; note = modified with 2'-Fluoro
  modified_base 12..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 50 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3..4: mod_base = OTHER; note = modified with 2'-Fluoro
  modified_base 5..6: mod_base = OTHER; note = modified with 2'-O-methyl
  modified_base 7..8: mod_base = OTHER; note = modified with 2'-Fluoro
  modified_base 9..10: mod_base = OTHER; note = modified with 2'-O-methyl
  modified_base 11..12: mod_base = OTHER; note = modified with 2'-Fluoro
  modified_base 13..14: mod_base = OTHER; note = modified with 2'-O-methyl
  modified_base 15..16: mod_base = OTHER; note = modified with 2'-Fluoro
  modified_base 17..18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttataatggtacttcctcaat

SEQ ID NO: 51 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 52 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 53 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 54 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..12: mod_base = OTHER; note = modified with 2'-O-methyl
  modified_base 13..14: mod_base = OTHER; note = modified with 2'-Fluoro
  modified_base 15..16: mod_base = OTHER; note = modified with 2'-O-methyl
  modified_base 17..18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 55 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 56 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..11: mod_base = OTHER; note = modified with 2'-O-methyl
  modified_base 12..13: mod_base = OTHER; note = modified with 2'-Fluoro
  modified_base 14..15: mod_base = OTHER; note = modified with 2'-O-methyl
  modified_base 16..17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 57 - RNA, 19 nt, synthetic construct
  modified_base 1..3: mod_base = OTHER; note = modified with phosphorothioate linker
  modified_base 1..3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 58 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 59 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 60 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 61 - RNA, 19 nt, synthetic construct
  modified_base 1..3: mod_base = OTHER; note = modified with phosphorothioate linker
  modified_base 1..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 62 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..11: mod_base = OTHER; note = modified with 2'-O-methyl
  modified_base 12: mod_base = OTHER; note = modified with 2'-Fluoro
  modified_base 13..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 63 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 64 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 65 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
tgaggaagtaccattataa

SEQ ID NO: 66 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 67 - DNA, 19 bp, synthetic construct
  modified_base 4..6: mod_base = OTHER; note = modified with phosphorothioate linker
  modified_base 4..5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 68 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12: mod_base = OTHER; note = modified with 2'-O-methyl
  modified_base 13: mod_base = OTHER; note = modified with 2'-Fluoro
  modified_base 14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttataatggtacttcctcaat

SEQ ID NO: 69 - DNA, 19 bp, synthetic construct
  modified_base 4..6: mod_base = OTHER; note = modified with phosphorothioate linker
  modified_base 4..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 10..11: mod_base = OTHER; note = modified with 2'-Fluoro
  modified_base 12..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 70 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3..4: mod_base = OTHER; note = modified with 2'-Fluoro
  modified_base 5..6: mod_base = OTHER; note = modified with 2'-O-methyl
  modified_base 7..8: mod_base = OTHER; note = modified with 2'-Fluoro
  modified_base 9..10: mod_base = OTHER; note = modified with 2'-O-methyl
  modified_base 11..12: mod_base = OTHER; note = modified with 2'-Fluoro
  modified_base 13..14: mod_base = OTHER; note = modified with 2'-O-methyl
  modified_base 15..16: mod_base = OTHER; note = modified with 2'-Fluoro
  modified_base 17..18: mod_base = OTHER; note = modified with 2'-O-methyl
  modified_base 19: mod_base = OTHER; note = modified with 2'-Fluoro
  modified_base 20..21: mod_base = OTHER; note = modified with 2'-O-methyl
  source 1..21: mol_type = other RNA
ttataatggtacttcctcaat

SEQ ID NO: 71 - DNA, 19 bp, synthetic construct
  modified_base 4..6: mod_base = OTHER; note = modified with phosphorothioate linker
  modified_base 4..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
tgaggaagtaccattataa

SEQ ID NO: 72 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 73 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 74 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..12: mod_base = OTHER; note = modified with 2'-O-methyl
  modified_base 13..14: mod_base = OTHER; note = modified with 2'-Fluoro
  modified_base 15..16: mod_base = OTHER; note = modified with 2'-O-methyl
  modified_base 17..18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 75 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5..6: mod_base = OTHER; note = modified with 2'-Fluoro
  modified_base 7..8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 76 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..11: mod_base = OTHER; note = modified with 2'-O-methyl
  modified_base 12..13: mod_base = OTHER; note = modified with 2'-Fluoro
  modified_base 14..15: mod_base = OTHER; note = modified with 2'-O-methyl
  modified_base 16..17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 77 - RNA, 19 nt, synthetic construct
  modified_base 4..6: mod_base = OTHER; note = modified with phosphorothioate linker
  modified_base 4: mod_base = OTHER; note = modified with 2'-Fluoro
  modified_base 5: mod_base = OTHER; note = modified with 2'-O-methyl
  modified_base 6..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 78 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 79 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 80 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 81 - RNA, 19 nt, synthetic construct
  modified_base 4..6: mod_base = OTHER; note = modified with phosphorothioate linker
  modified_base 4..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 9..10: mod_base = OTHER; note = modified with 2'-Fluoro
  modified_base 11..19: mod_base = OTHER; note = modified with 2'-O-methyl
  source 1..19: mol_type = other RNA
tgaggaagtaccattataa

SEQ ID NO: 82 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..11: mod_base = OTHER; note = modified with 2'-O-methyl
  modified_base 12: mod_base = OTHER; note = modified with 2'-Fluoro
  modified_base 13..14: mod_base = OTHER; note = modified with 2'-O-methyl
  modified_base 15: mod_base = OTHER; note = modified with 2'-Fluoro
  modified_base 16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttataatggtacttcctcaat

SEQ ID NO: 83 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5..9: mod_base = OTHER; note = modified with 2'-Fluoro
  modified_base 10..19: mod_base = OTHER; note = modified with 2'-O-methyl
  source 1..19: mol_type = other RNA
tgaggaagtaccattataa

SEQ ID NO: 84 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11..15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttataatggtacttcctcaat

SEQ ID NO: 85 - RNA, 19 nt, synthetic construct
  modified_base 1..3: mod_base = OTHER; note = modified with phosphorothioate linker
  modified_base 1..6: mod_base = OTHER; note = modified with 2'-O-methyl
  modified_base 7..9: mod_base = OTHER; note = modified with 2'-Fluoro
  modified_base 10..19: mod_base = OTHER; note = modified with 2'-O-methyl
  source 1..19: mol_type = other RNA
aggccacattcacgtataa

SEQ ID NO: 86 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 87 - DNA, 19 bp, synthetic construct
  modified_base 1..3: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 88 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 89 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 90 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 91 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 92 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..8: mod_base = OTHER; note = modified with 2'-Fluoro
  modified_base 9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 93 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 94 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 95 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 96 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3..4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 97 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 98 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 99 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 100 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15..16: mod_base = OTHER; note = modified with 2'-O-methyl
  modified_base 17..18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 101 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 102 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 103 - RNA, 19 nt, synthetic construct
  modified_base 1..3: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4..6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
aggccacattcacgtataa

SEQ ID NO: 104 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttatacgtgaatgtggcctgt

SEQ ID NO: 105 - DNA, 19 bp, synthetic construct
  modified_base 1..3: mod_base = OTHER; note = modified with phosphorothioate linker
  modified_base 1..3: mod_base = OTHER; note = modified with 2'-O-methyl
  modified_base 4: mod_base = OTHER; note = modified with 2'-Fluoro
  modified_base 5..6: mod_base = OTHER; note = modified with 2'-O-methyl
  modified_base 7..8: mod_base = OTHER; note = modified with 2'-Fluoro
  modified_base 10: mod_base = OTHER; note = modified with 2'-Fluoro
  modified_base 11..19: mod_base = OTHER; note = modified with 2'-O-methyl
  source 1..19: mol_type = other DNA
  modified_base 1..8: mod_base = OTHER; note = RNA
  modified_base 9: mod_base = OTHER; note = DNA
  modified_base 10..19: mod_base = OTHER; note = RNA
aggccacattcacgtataa

SEQ ID NO: 106 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8..9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..13: mod_base = OTHER; note = modified with 2'-O-methyl
  modified_base 14: mod_base = OTHER; note = modified with 2'-Fluoro
  modified_base 15..16: mod_base = OTHER; note = modified with 2'-O-methyl
  modified_base 17: mod_base = OTHER; note = modified with 2'-Fluoro
  modified_base 18..21: mod_base = OTHER; note = modified with 2'-O-methyl
  source 1..21: mol_type = other RNA
ttatacgtgaatgtggcctgt

SEQ ID NO: 107 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
tgaggaagtaccattataa

SEQ ID NO: 108 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17..21: mod_base = OTHER; note = modified with 2'-O-methyl
  source 1..21: mol_type = other RNA
ttataatggtacttcctcaat

SEQ ID NO: 109 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
tgaggaagtaccattataa

SEQ ID NO: 110 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17..21: mod_base = OTHER; note = modified with 2'-O-methyl
  source 1..21: mol_type = other RNA
ttataatggtacttcctcaat

SEQ ID NO: 111 - RNA, 19 nt, synthetic construct
  source 1..19: mol_type = other RNA
ctgtcaaacccggtaattt

SEQ ID NO: 112 - RNA, 21 nt, synthetic construct
  source 1..21: mol_type = other RNA
aaattaccgggtttgacagtt

SEQ ID NO: 113 - DNA, 19 bp, synthetic construct
  modified_base 1..3: mod_base = OTHER; note = modified with phosphorothioate linker
  modified_base 1..6: mod_base = OTHER; note = modified with 2'-O-methyl
  modified_base 7..8: mod_base = OTHER; note = modified with 2'-Fluoro
  modified_base 9: mod_base = OTHER; note = DNA
  modified_base 10..19: mod_base = OTHER; note = modified with 2'-O-methyl
  source 1..19: mol_type = other DNA
  modified_base 1..8: mod_base = OTHER; note = RNA
  modified_base 10..19: mod_base = OTHER; note = RNA
ctgtcaaacccggtaattt

SEQ ID NO: 114 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2..3: mod_base = OTHER; note = modified with 2'-Fluoro
  modified_base 4..5: mod_base = OTHER; note = modified with 2'-O-methyl
  modified_base 6..7: mod_base = OTHER; note = modified with 2'-Fluoro
  modified_base 8..9: mod_base = OTHER; note = modified with 2'-O-methyl
  modified_base 10..11: mod_base = OTHER; note = modified with 2'-Fluoro
  modified_base 12..13: mod_base = OTHER; note = modified with 2'-O-methyl
  modified_base 14..15: mod_base = OTHER; note = modified with 2'-Fluoro
  modified_base 16..17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 115 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ctgtcaaacccggtaattt

SEQ ID NO: 116 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 117 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ctgtcaaacccggtaattt

SEQ ID NO: 118 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1..2: mod_base = OTHER; note = modified with 2'-O-methyl
  modified_base 3: mod_base = OTHER; note = modified with 2'-Fluoro
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 119 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ctgtcaaacccggtaattt

SEQ ID NO: 120 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 121 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ctgtcaaacccggtaattt

SEQ ID NO: 122 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 123 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
ctgtcaaacccggtaattt

SEQ ID NO: 124 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 125 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
ctgtcaaacccggtaattt

SEQ ID NO: 126 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 127 - RNA, 19 nt, synthetic construct
  modified_base 4..6: mod_base = OTHER; note = modified with phosphorothioate linker
  modified_base 4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7: mod_base = OTHER; note = modified with 2'-Fluoro
  modified_base 8: mod_base = OTHER; note = modified with 2'-O-methyl
  modified_base 9: mod_base = OTHER; note = modified with 2'-Fluoro
  modified_base 10: mod_base = OTHER; note = modified with 2'-O-methyl
  modified_base 11: mod_base = OTHER; note = modified with 2'-Fluoro
  modified_base 12..19: mod_base = OTHER; note = modified with 2'-O-methyl
  source 1..19: mol_type = other RNA
ctgtcaaacccggtaattt

SEQ ID NO: 128 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4: mod_base = OTHER; note = modified with 2'-Fluoro
  modified_base 5: mod_base = OTHER; note = modified with 2'-O-methyl
  modified_base 6: mod_base = OTHER; note = modified with 2'-Fluoro
  modified_base 7: mod_base = OTHER; note = modified with 2'-O-methyl
  modified_base 8: mod_base = OTHER; note = modified with 2'-Fluoro
  modified_base 9: mod_base = OTHER; note = modified with 2'-O-methyl
  modified_base 10: mod_base = OTHER; note = modified with 2'-Fluoro
  modified_base 11: mod_base = OTHER; note = modified with 2'-O-methyl
  modified_base 12: mod_base = OTHER; note = modified with 2'-Fluoro
  modified_base 13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17..21: mod_base = OTHER; note = modified with 2'-O-methyl
  source 1..21: mol_type = other RNA
aaattaccgggtttgacagtt

SEQ ID NO: 129 - RNA, 19 nt, synthetic construct
  source 1..19: mol_type = other RNA
gccactagtctctagtcaa

SEQ ID NO: 130 - RNA, 21 nt, synthetic construct
  source 1..21: mol_type = other RNA
ttgactagagactagtggctt

SEQ ID NO: 131 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
gccactagtctctagtcaa

SEQ ID NO: 132 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttgactagagactagtggctt

SEQ ID NO: 133 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ctgtcaaacccggtaattt

SEQ ID NO: 134 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
aaattaccgggtttgacagtt

SEQ ID NO: 135 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
gccactagtctctagtcaa

SEQ ID NO: 136 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttgactagagactagtggctt

SEQ ID NO: 137 - DNA, 24 bp, synthetic construct
  source 1..24: mol_type = other DNA
ctctgatcatctgaccaaaactca

SEQ ID NO: 138 - DNA, 21 bp, synthetic construct
  source 1..21: mol_type = other DNA
caacccagacatatccacctc

SEQ ID NO: 139 - DNA, 24 bp, synthetic construct
  modified_base 1: mod_base = OTHER; note = modified with 5'6-FAM
  modified_base 9..10: mod_base = OTHER; note = modified with ZEN
  modified_base 24: mod_base = OTHER; note = modified with 3' Iowa Black FQ
  source 1..24: mol_type = other DNA
tggcaagcatcctgtactgtcctg

SEQ ID NO: 140 - DNA, 19 bp, synthetic construct
  source 1..19: mol_type = other DNA
acatcgctcagacaccatg

SEQ ID NO: 141 - DNA, 22 bp, synthetic construct
  source 1..22: mol_type = other DNA
tgtagttgaggtcaatgaaggg

SEQ ID NO: 142 - DNA, 25 bp, synthetic construct
  modified_base 1: mod_base = OTHER; note = modified with 5'6-FAM
  modified_base 9..10: mod_base = OTHER; note = modified with ZEN
  modified_base 25: mod_base = OTHER; note = modified with 3' Iowa Black FQ
  source 1..25: mol_type = other DNA
aaggtcggagtcaacggatttggtc

SEQ ID NO: 143 - DNA, 26 bp, synthetic construct
  source 1..26: mol_type = other DNA
acaggtacgttaatagttaatagcgt

SEQ ID NO: 144 - DNA, 22 bp, synthetic construct
  source 1..22: mol_type = other DNA
acattgcagcagtacgcacaca

SEQ ID NO: 145 - DNA, 26 bp, synthetic construct
  source 1..26: mol_type = other DNA
acactagccatccttactgcgcttcg

SEQ ID NO: 146 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ggaggctgtaggcataaaa

SEQ ID NO: 147 - RNA, 21 nt, synthetic construct
  modified_base 1..3: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttttatgcctacagcctcctt

SEQ ID NO: 148 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ggaggctgtaggcataaaa

SEQ ID NO: 149 - RNA, 21 nt, synthetic construct
  modified_base 1: mod_base = OTHER; note = modified with 5'-E-vynylphosphonate
  modified_base 1..3: mod_base = OTHER; note = modified with phosphorothioate linker
  modified_base 5..7: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttttatgcctacagcctcctt

SEQ ID NO: 150 - RNA, 19 nt, synthetic construct
  modified_base 1..3: mod_base = OTHER; note = modified with phosphorothioate linker
  modified_base 1..4: mod_base = OTHER; note = modified with 2'-O-methyl
  modified_base 5: mod_base = OTHER; note = modified with 2'-Fluoro
  modified_base 6: mod_base = OTHER; note = modified with 2'-O-methyl
  modified_base 7..10: mod_base = OTHER; note = modified with 2'-Fluoro
  modified_base 11..19: mod_base = OTHER; note = modified with 2'-O-methyl
  source 1..19: mol_type = other RNA
ggaggctgtaggcataaaa

SEQ ID NO: 151 - RNA, 21 nt, synthetic construct
  modified_base 1: mod_base = OTHER; note = modified with 5'-phosphorothioate
  modified_base 1..3: mod_base = OTHER; note = modified with phosphorothioate linker
  modified_base 5..7: mod_base = OTHER; note = modified with phosphorothioate linker
  modified_base 19..21: mod_base = OTHER; note = modified with phosphorothioate linker
  modified_base 1: mod_base = OTHER; note = modified with 2'-O-methyl
  modified_base 2: mod_base = OTHER; note = modified with 2'-Fluoro
  modified_base 3: mod_base = OTHER; note = modified with 2'-O-methyl
  modified_base 4..5: mod_base = OTHER; note = modified with 2'-Fluoro
  modified_base 6..7: mod_base = OTHER; note = modified with 2'-O-methyl
  modified_base 8..9: mod_base = OTHER; note = modified with 2'-Fluoro
  modified_base 10..13: mod_base = OTHER; note = modified with 2'-O-methyl
  modified_base 14: mod_base = OTHER; note = modified with 2'-Fluoro
  modified_base 15: mod_base = OTHER; note = modified with 2'-O-methyl
  modified_base 16: mod_base = OTHER; note = modified with 2'-Fluoro
  modified_base 17: mod_base = OTHER; note = modified with 2'-O-methyl
  modified_base 18: mod_base = OTHER; note = modified with 2'-Fluoro
  modified_base 19..21: mod_base = OTHER; note = modified with 2'-O-methyl
  source 1..21: mol_type = other RNA
ttttatgcctacagcctcctt

SEQ ID NO: 152 - DNA, 22 bp, synthetic construct
  source 1..22: mol_type = other DNA
ccgtctgtgccttctcatctgc

SEQ ID NO: 153 - DNA, 23 bp, synthetic construct
  source 1..23: mol_type = other DNA
accaatttatgcctacagcctcc

SEQ ID NO: 154 - DNA, 20 bp, synthetic construct
  source 1..20: mol_type = other DNA
catcactgccacccagaaga

SEQ ID NO: 155 - DNA, 20 bp, synthetic construct
  source 1..20: mol_type = other DNA
cctgcttcaccaccttcttg